(19) United States
(12) Patent Application Publication
(10) Pub. No.: US 2013/0211408 A1
(43) Pub. Date: Aug. 15, 2013

(54) Curved Arthroscopic Burr and Measurement Instrumentation

(71) Applicants: IMDS CORPORATION, (US); Jens Kather, (US); Michael Schueler, (US)

(72) Inventors: Jens Kather, Benglen (CH); Daniel F. Justin, Orlando, FL (US); Joshua A. Butters, Chandler, AZ (US); Jeffery D. Arnett, Gilbert, AZ (US); Nicholas M. Slater, Chandler, AZ (US); Melissa D. Scott, Flanders, NJ (US); Bradford J. Coale, Flanders, NJ (US)

(73) Assignees: Jens Kather, Benglen (CH); IMDS CORPORATION, Logan, UT (US); Michael Schueler, Kreuszlingen (CH)

(21) Appl. No.: 13/763,243

(22) Filed: Feb. 8, 2013

Related U.S. Application Data
(60) Provisional application No. 61/596,590, filed on Feb. 8, 2012.
(60) Provisional application No. 61/655,391, filed on Jun. 4, 2012.
(60) Provisional application No. 61/722,940, filed on Nov. 6, 2012.

(51) Int. Cl. A61B 17/16

(52) U.S. Cl. CPC A61B 17/1604
USPC 606/83

(57) ABSTRACT

A tissue resection device includes a housing, a drive shaft, and a set of cutting inserts arranged in sequence. The cutting inserts are connected in the sequence by off-axis torque couplings so that each cutting insert may lie at an angle to an adjacent cutting insert. Each cutting insert includes at least one cutting edge, and the cutting edges in sequence form a cutting profile which may include a combination of straight and curved sections. The housing may be curved. Rotation of the drive shaft rotates the set of cutting inserts to form a cutting burr.

### CROSS-REFERENCE TO RELATED APPLICATIONS

This application claims the benefit of:

U.S. Provisional Patent Application No. 61/596,590, filed Feb. 8, 2012, entitled CURVED ARTHROSCOPIC BURR AND MEASUREMENT INSTRUMENTATION, Attorney's docket no. KAT-1 PROV, which is pending.

U.S. Provisional Patent Application No. 61/655,391, filed Jun. 4, 2012, entitled CURVED ARTHROSCOPIC BURR AND MEASUREMENT INSTRUMENTATION, Attorney's docket no. KAT-2 PROV, which is pending.

U.S. Provisional Patent Application No. 61/722,940, filed Nov. 6, 2012, entitled CURVED BURR SURGICAL INSTRUMENT, Attorney's docket no. KAT-4 PROV, which is pending.

The above-referenced documents are hereby incorporated by reference in their entirety.

### BACKGROUND

The present disclosure relates to surgical instruments, such as arthroscopic surgical instruments. The principles herein are applicable in tissue removal applications, whether arthroscopic, laparoscopic, endoscopic, or open, including but not limited to: foot, ankle, knee, hip, pelvis, spine, ribs, shoulder, elbow, wrist, hand, craniomaxillofacial, etc.

Straight or spherical rigid cutting instruments are not well suited to creating a smooth anatomic radius of curvature with minimal manipulation. Therefore, with these tools, it is up to the surgeon to sculpt a three-dimensional (3D) anatomic surface by manipulating the cutter over the treated surface, without unintentionally removing too much tissue.

It is desirable to provide a more efficient means of tissue removal, including removal of sclerotic bone, in order to reduce operating time. The disclosed examples are capable of removing tissue on multiple surfaces at once, creating a smooth uniform surface with minimal manipulation of the instrument. The instruments described herein may automatically re-establish a proper anatomic profile to the treated surface by matching the natural anatomic profile of the tissue. The instruments described herein are capable of producing 3D shaping with simple two-dimensional (2D) manipulation of the instruments. The instruments and methods described herein may significantly reduce operating time and produce more uniform results.

The tissue resection devices or burr tools disclosed herein are capable of matching the natural anatomic curvature of a tissue. In one example, the tissue resection device includes an outer housing, central flexible member, cutting elements, and bearing elements. The outer housing may be curved to approximately match the geometry of a tissue. The outer housing or sheath may have at least one cutout or window through which the cutting elements, inserts, or burrs are exposed to effect the tissue resection. The cutout or window in the outer housing may be adjusted to vary the amount of burr exposure through the window to vary the amount/depth of tissue that is removed in a single pass of the instrument. In this manner, the window may act like a depth stop to provide extra control and precision over tissue removal and prevent unintended tissue removal. In some examples, the depth stop may allow a substantially uniform depth cut along the curved burr portion of the resection device. For example, the window in the outer sheath may be sized to allow the burrs to project about 1 mm from the window. In other examples, the burrs may project more or less than 1 mm. In yet other examples, the user may selectively adjust how much the burrs project from the window. In this manner, the user may control the depth of the tissue cut in a single pass and prevent the resection device from cutting away too much tissue.

In some examples disclosed herein, the cutting inserts, elements, or burrs are circular members that include a number of cutting arms with sharp edges that are designed to cut tissue, such as bone. The number of cutting arms may be varied to remove more or less tissue in a single revolution of the burr. In one example, the cutting insert has three arms. The cutting elements may also include a central hole through which a central flexible member may pass. The burrs may be slid over the central flexible member to create a flexible cutting assembly. In this example, the burrs may act like beads on a necklace moving and flexing with the central member. The burr elements may also possess mating teeth that interface with adjacent burrs to form a stack. The mating teeth allow torque to be transmitted through the series or stack of mating burrs. In some examples, the central flexible member may, or may not, be required to provide torque to the system as the burrs themselves may transmit the torque required for cutting.

In some applications of the disclosed technology, the efficacy of the tissue removal procedure may be evaluated by removing all instruments and articulating the affected joint post-resection to ensure impingement-free motion. Direct visualization, medical imaging, palpation, or other assessment means may also be used to evaluate the effectiveness of the tissue removal. Operative time may be further reduced by using measurement devices that serve as anatomic templates for the treated region. The measurement devices disclosed herein provide an anatomic basis for evaluation of tissue removal, without the need to articulate the joint and directly test range of motion, or resort to medical imaging, or the like.

In an example, a tissue resection device includes a housing extending between proximal and distal ends, a portion of the housing extending along an arcuate path forming an arcuate housing portion, the housing including a housing window; a drive shaft; and a plurality of cutting inserts coupled together in a sequence extending along the arcuate path and partially received in the housing window, the proximal most cutting insert coupled to the drive shaft, wherein each cutting insert includes at least one cutting edge and wherein each cutting insert is non-cylindrical.

In another example, a tissue resection device includes a housing extending between proximal and distal ends, a portion of the housing extending along an arcuate path forming an arcuate housing portion, the housing including a housing window; a drive shaft; and a plurality of cutting inserts coupled together in a sequence extending along the arcuate path and partially received in the housing window, the proximal most cutting insert coupled to the drive shaft, wherein each cutting insert includes at least one cutting edge and wherein each cutting insert is differently shaped than each of the other cutting inserts.

In yet another example, a tissue resection device includes a housing extending between proximal and distal ends, a portion of the housing extending along an arcuate path forming an arcuate housing portion, the housing including a housing window; a drive shaft; and a plurality of cutting inserts coupled together in a sequence extending along the arcuate path and partially received in the housing window, the proximal most cutting insert coupled to the drive shaft, wherein each cutting insert includes at least one cutting edge, and wherein the cutting edges form a cutting profile which includes at least one straight section and at least one curved section.

### BRIEF DESCRIPTION OF THE DRAWINGS

While examples of the present technology have been shown and described in detail below, it will be clear to the person skilled in the art that variations, changes and modifications may be made without departing from its scope. As such, that which is set forth in the following description and accompanying drawings is offered by way of illustration only and not as a limitation. The actual scope of the invention is intended to be defined by the following claims, along with the full range of equivalents to which such claims are entitled.

In the following Detailed Description, various features are grouped together in several examples for the purpose of streamlining the disclosure. This method of disclosure is not to be interpreted as reflecting an intention that examples of the technology require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example.

Identical reference numerals do not necessarily indicate an identical structure. Rather, the same reference numeral may be used to indicate a similar feature or a feature with similar functionality. Not every feature of each example is labeled in every figure in which that example appears, in order to keep the figures clear. Similar reference numbers (e.g., those that are identical except for the first numeral) are used to indicate similar features in different examples.

FIG. 1 is a side view of a tissue resection device;

FIG. 2A is an isometric detail view of a portion of the tissue resection device of FIG. 1; and FIG. 2B is a side detail view of the portion of the tissue resection device of FIG. 1 shown in FIG. 2A, with an outer sheath removed;

FIG. 3 is an isometric view of a cutting insert of the tissue resection device of FIG. 1;

FIG. 4 is an isometric view of several cutting inserts in a stacked arrangement;

FIG. 5 is a side view of a measurement device;

FIG. 6 is a side detail view of a portion of the measurement device of FIG. 5

FIG. 7 is a side detail view of a portion of another measurement device;

FIG. 8 is an isometric view of the tissue resection device of FIG. 1 in use against a femoral neck;

FIG. 9 is an isometric detail view of the measurement device of FIG. 5 in use against a femoral neck and head;

FIG. 10 is a side detail view of the measurement device of FIG. 7 in use against a tissue surface;

FIG. 11A is a side view of another tissue resection device; FIG. 11B is a detail view of a portion of the tissue resection device of FIG. 11A in a deployed condition; and FIG. 11C is another detail view of a portion of the tissue resection device of FIG. 11A in a retracted condition;

FIG. 12 is a side detail view of a portion of yet another measurement device;

FIG. 13 is a side detail view of a portion of yet another measurement device;

FIG. 14 is an isometric detail view of a portion of the tissue resection device of FIG. 11A and a guide tool in use against a femoral neck and head;

FIG. 15 is a side detail view of a portion of the measurement device of FIG. 12 in use against a femoral neck and head;

FIG. 16 is a side detail view of a portion of yet another tissue resection device;

FIG. 17 is a side detail view of a portion of yet another tissue resection device;

FIG. 18 is an isometric view of the tissue resection device of FIG. 1 and a stabilization arm;

FIG. 19 is a side view of an arcuate portion of yet another tissue resection device with an outer sheath and some of the cutting inserts removed;

FIG. 20A is a cross-sectional view of a distal end of yet another tissue resection device; FIG. 20B is a side view of a cannulated flexible member of the tissue resection device of FIG. 20A; FIG. 20C is a perspective view of a cutting insert of the tissue resection device of FIG. 20A engaged with the flexible member of FIG. 20B; and FIG. 20D is a side view of the cutting insert of FIG. 20C;

FIG. 21 is a perspective view of the cutting insert of FIG. 20C;

FIG. 22 is a perspective view of a distal end of yet another tissue resection device;

FIG. 23 is a perspective view of the tissue resection device of FIG. 22 with an end cover removed;

FIG. 24 is a perspective view of the distal end of yet another tissue resection device with an outer sheath removed;

FIG. 25 is a perspective view of the tissue resection device of FIG. 24 with an end cover removed;

FIG. 26 is a perspective view of the distal end of yet another tissue resection device with an outer sheath and an end cover removed;

FIG. 27A is a side cross-sectional view of the tissue resection device of FIG. 26; FIG. 27B is a perspective view of a cutting insert of the tissue resection device of FIG. 26; FIG. 27C is a perspective view of the cutting insert of FIG. 27B; and FIG. 27D is a table of exemplary connection shapes which may be used with the cutting inserts of the present disclosure;

FIG. 28 is a side view of yet another tissue resection device with the cutting inserts exposed on a convex portion of the distal end of the tip;

FIG. 29 is a side view of yet another tissue resection device with the cutting inserts exposed on convex and concave portions of the distal end of the tip;

FIG. 30A is a side view of yet another tissue resection device; FIG. 30B is a side view of yet another tissue resection device; FIG. 30C is a side view of yet another tissue resection device; and FIG. 30D is a side view of yet another tissue resection device;

FIG. 31A is an isometric view of yet another tissue resection device; and FIG. 3 IB is a side view of the tissue resection device of FIG. 31A;

FIG. 32A is a top view of the tissue resection device of FIG. 31A; and FIG. 32B is a bottom view of the tissue resection device of FIG. 31A;

FIG. 33 is an isometric exploded view of the tissue resection device of FIG. 31A;

FIG. 34 is a side cross-sectional view of the tissue resection device of FIG. 31A taken along section line 4-4 in FIG. 32A;

FIG. 35A is a top view of a housing of the tissue resection device of FIG. 31A; FIG. 35B is a bottom view of the housing of FIG. 35A; FIG. 35C is a side view of the housing of FIG. 35A; FIG. 35D is a side cross-sectional view of the housing of FIG. 35A taken along section line 5-5 in FIG. 35A;

FIG. 36A is an isometric view of a cutting insert of the tissue resection device of FIG. 31A; FIG. 36B is a side view of the cutting insert of FIG. 36A; FIG. 36C is an end view of the cutting insert of FIG. 36A;

FIG. 37A is an isometric view of another cutting insert of the tissue resection device of FIG. 31A; FIG. 37B is a side view of the cutting insert of FIG. 37A; FIG. 37C is a side cross-sectional view of the cutting insert of FIG. 37A; and FIG. 37D is an end view of the cutting insert of FIG. 37A;

FIG. 38 is a side view of a cutting assembly of cutting inserts of the tissue resection device of FIG. 31A;

FIG. 39 is a line depicting a profile cut by the cutting assembly of the tissue resection device of FIG. 31A;

FIG. 40 is a side view of cutting inserts of yet another tissue resection device;

FIG. 41 is a side view of cutting inserts of yet another tissue resection device;

FIG. 42 is a side view of cutting inserts of yet another tissue resection device;

FIG. 43 is a side view of cutting inserts of yet another tissue resection device;

FIG. 44 is a side view of cutting inserts, bushings and drive shaft of yet another tissue resection device;

FIG. 45 is a side view of cutting inserts, bushings and drive shaft of yet another tissue resection device; and

FIGS. 46-55 are lines depicting profiles which may be cut by other tissue resection devices.

### DETAILED DESCRIPTION

Standard medical planes of reference and descriptive terminology are employed in this specification. A sagittal plane divides a body into right and left portions. A mid-sagittal plane divides the body into bilaterally symmetric right and left halves. A coronal plane divides a body into anterior and posterior portions. A transverse plane divides a body into superior and inferior portions. Anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body. Ipsilateral means on the same side of the body. Contralateral means on the opposite side of the body. These descriptive terms may be applied to an animate or inanimate body.

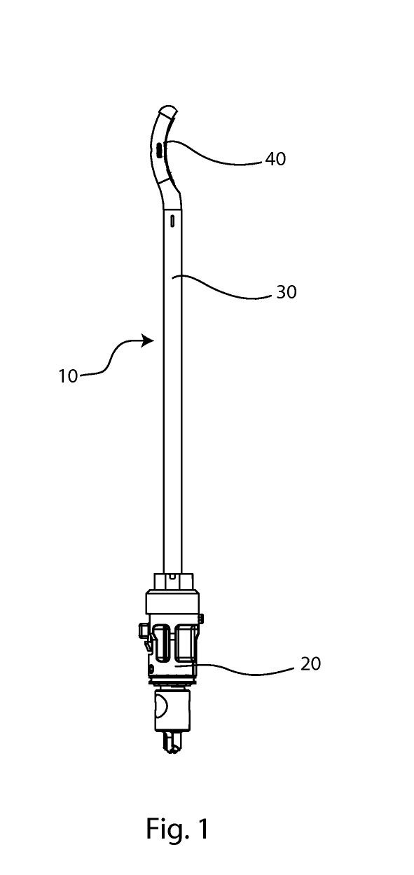

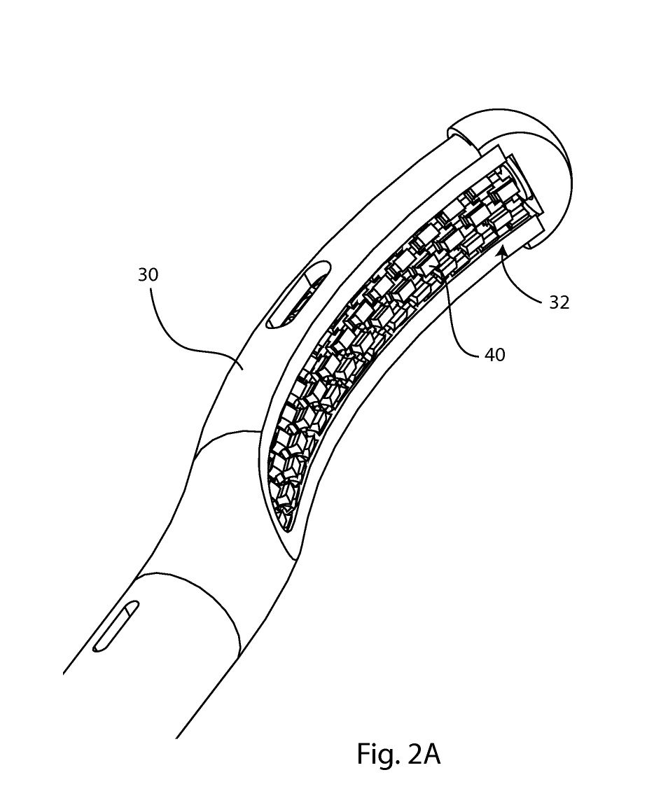

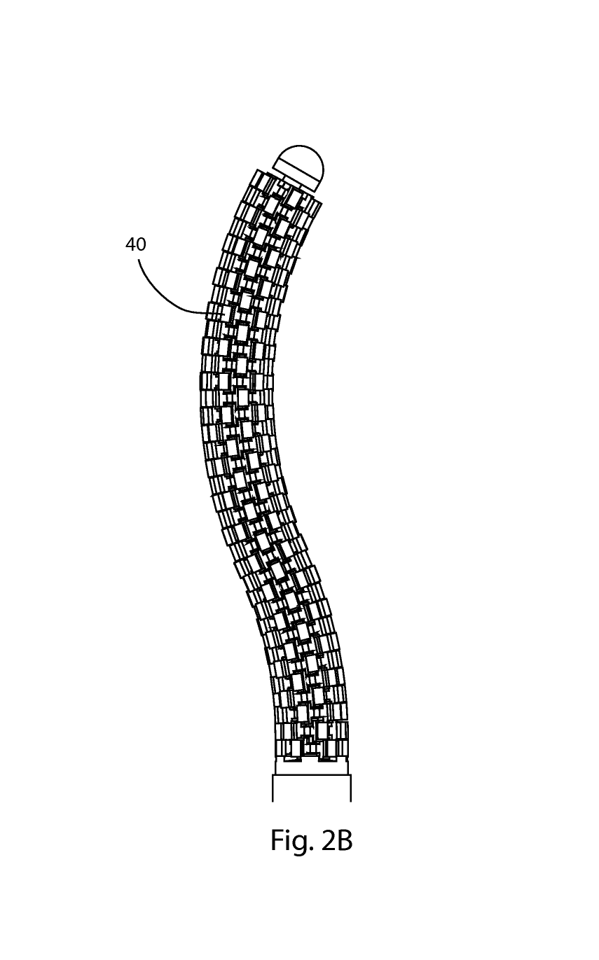

Referring to FIG. 1, a tissue resection device 10 may include a proximal drive hub 20, an outer tube or sheath 30, and distal cutting inserts 40. As shown in FIG. 2A, the cutting inserts 40 are exposed through a window 32 in the sheath 30 yet retained within the diameter of the outer sheath 30. The cutting inserts 40 may rotate within the outer sheath 30. FIG. 2B illustrates the cutting inserts 40 interlocking together and adapting to a curvature.

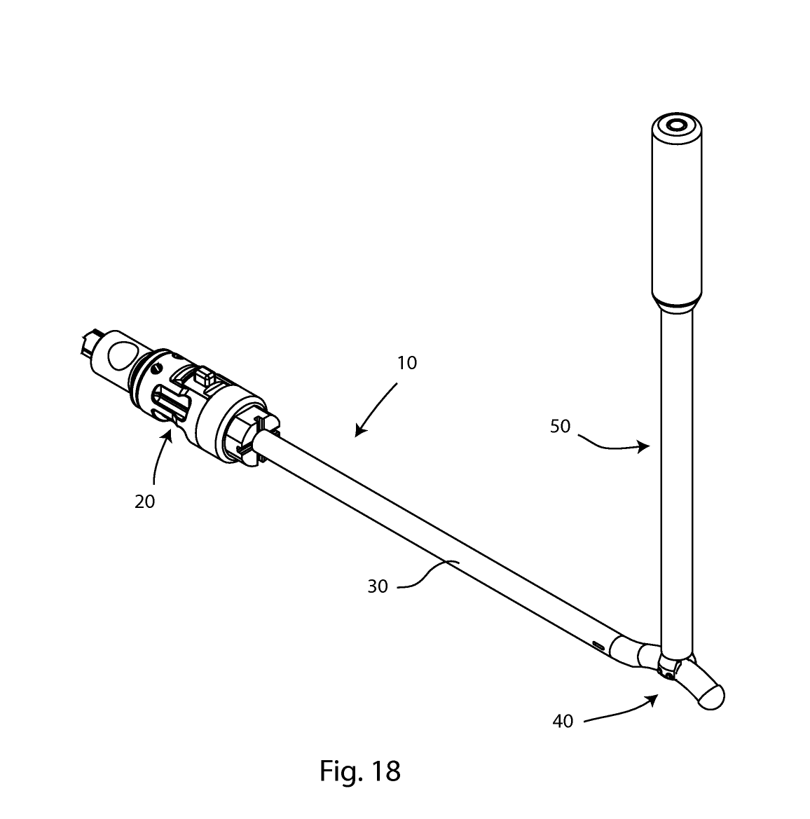

In some examples, the tissue resection device 10 may also include an optional stabilization arm 50 that may attach near the distal end of the instrument to provide greater force and control while manipulating the instrument. FIG. 18 illustrates one example of a tissue resection device 10 that includes an optional stabilization arm 50. The stabilization arm 50 may couple to the sheath 30 between the hub 20 and the distal end of the resection device 10. For example, the stabilization arm may couple to the sheath 30 near the cutting inserts 40. The stabilization arm may extend oblique, transverse, or perpendicular to the sheath 30. When coupled to the sheath 30, the arm may lie in the same plane as the arc of curvature of the distal cutting portion, or at another angle. The stabilization arm 50 may be selectively detached or attached to the tissue resection device 10, or may be an integrally formed member of the tissue resection device 10.

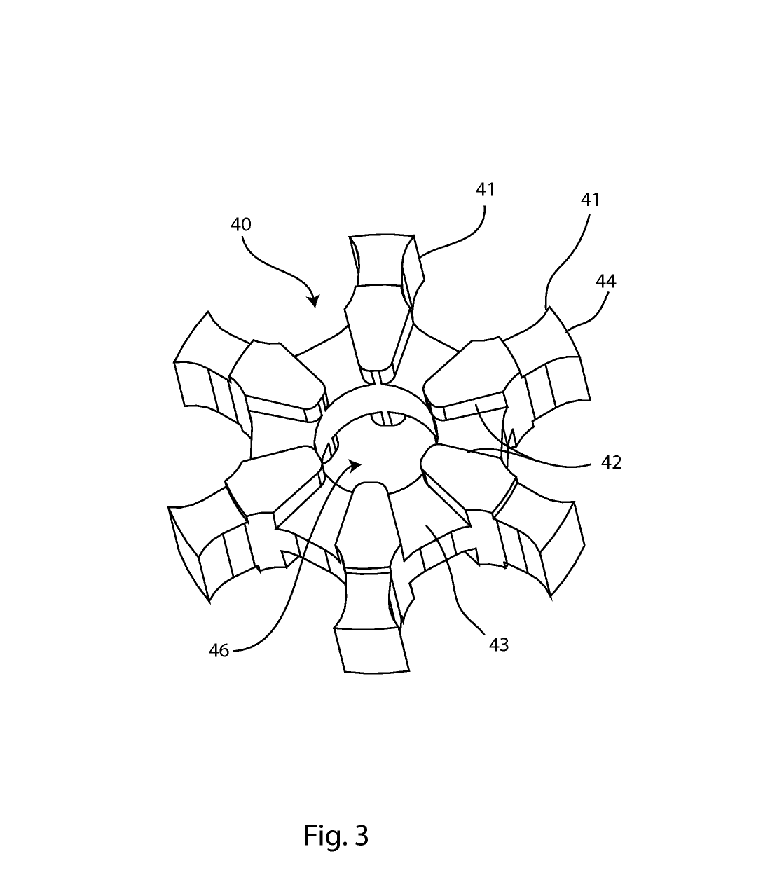

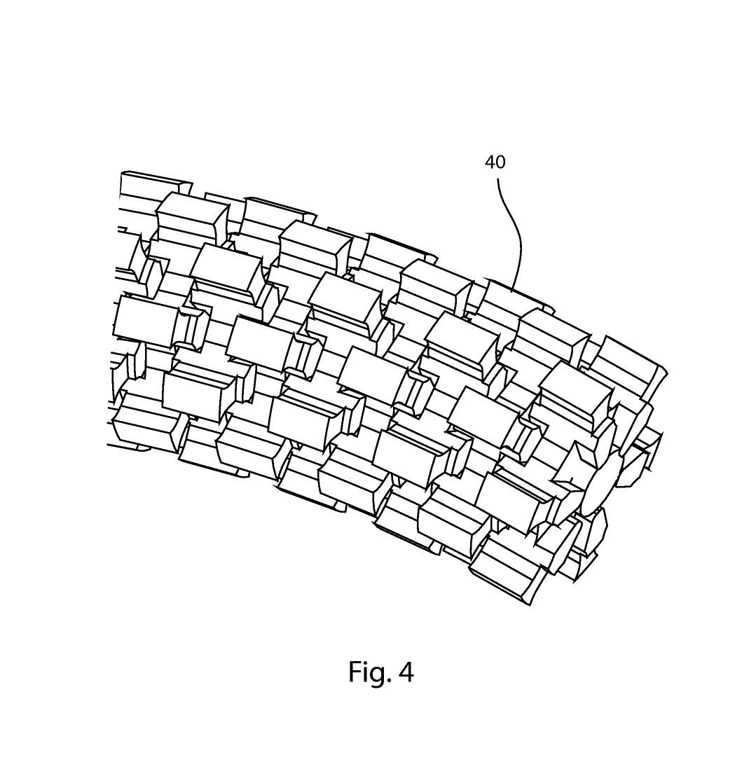

As shown in FIG. 3, the cutting insert 40 may have a plurality of arms 44 and cutting edges 41 radially arranged about a central aperture 46. The cutting inserts 40 may mesh together, interlock, or overlap each other, by fitting into the interlock recesses 43 of adjacent cutting inserts 40 as demonstrated in FIG. 4. In this example, each cutting insert 40 may be driven to rotate through their respective mating surfaces 42 by adjacent cutting inserts 40. Thus, in this example, the cutting inserts 40 are constructed such that each insert drives the next one. The illustrated interconnection between adjacent cutting inserts 40 is an example of a torque coupling which transmits torque between two adjacent cutting inserts which are set at an angle to each other.

In other examples, torque may be transmitted by other means to each cutting insert 40, such as by a central flexible member, drive shaft, or cable. The flexible member may be a stranded flexible metallic cable, flexible wire, a flexible laser-cut puzzle piece shaft, spring, flexible polymeric shaft/wire, or the like.

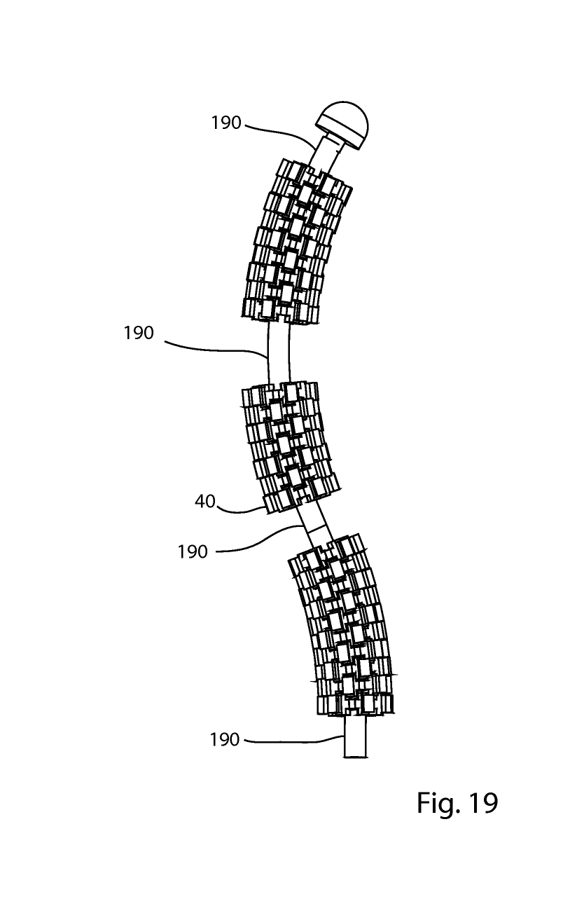

FIG. 19 illustrates a portion of the distal end of another tissue resection device utilizing a flexible cable 190 to drive cutting inserts 40. Some of the cutting inserts 40 have been removed to better visualize how the flexible cable 190 may interact with the cutting inserts 40. In some examples, the cutting inserts 40 may be welded to the flexible cable 190 and driven by the cable 190 directly. In other examples, the flexible cable may be shaped to interact with complimentary shapes formed in each cutting insert 40 such that the flexible cable 190 drives each cutting insert directly. For example, the cable may have protrusions or depressions formed therein and shaped to act with complimentary shaped protrusions or depressions formed in each cutting insert 40. In yet other examples, there may be a mix of some cutting inserts 40 that are driven by adjacent cutting inserts 40 and other cutting inserts 40 that are driven directly by the central flexible drive shaft or cable 190, in any proportion, ratio, or number.

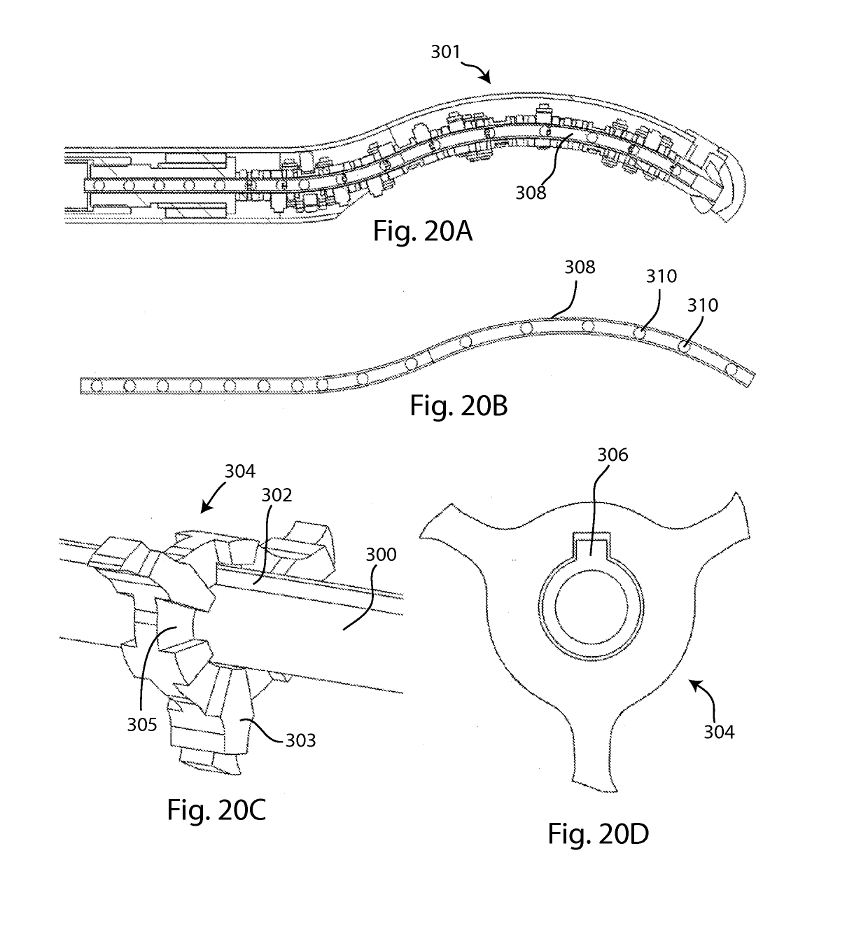

FIGS. 20A-20D illustrate one example of a tissue removal device 301 with a flexible member 300 with a key 302 shaped to interact with a slot 306 formed in a suitable cutting insert 304. The flexible member 300 imparts torque to the cutting insert 304 through the key 302 engaged in the slot 306 of the cutting insert. The cutting insert 304 may also impart torque to adjacent cutting inserts 304 through projections 303 interacting with recesses 305 such that adjacent cutting inserts 304 do not need a slot 306, similar to the cutting insert shown in FIG. 21.

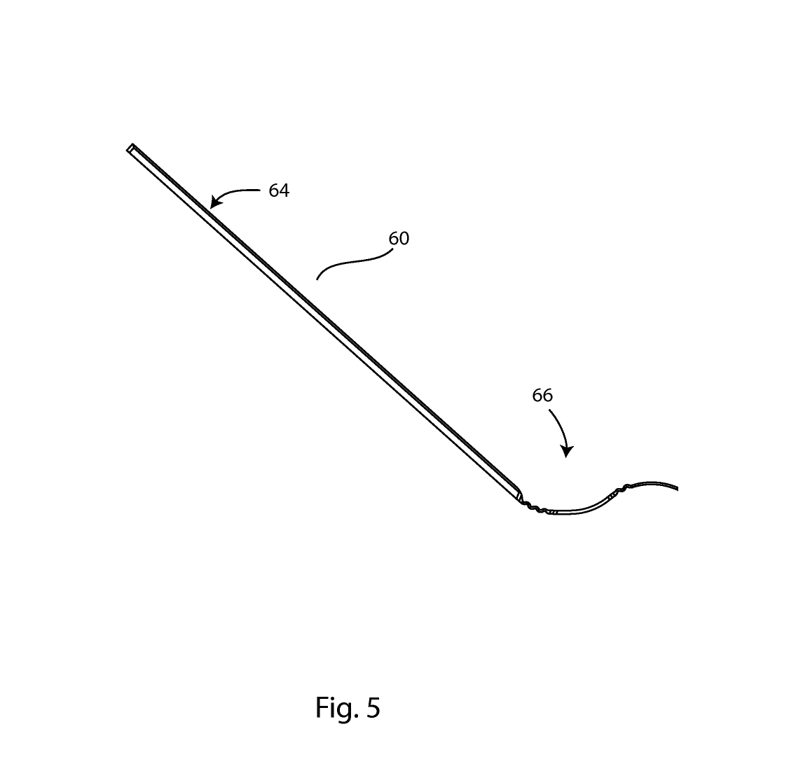

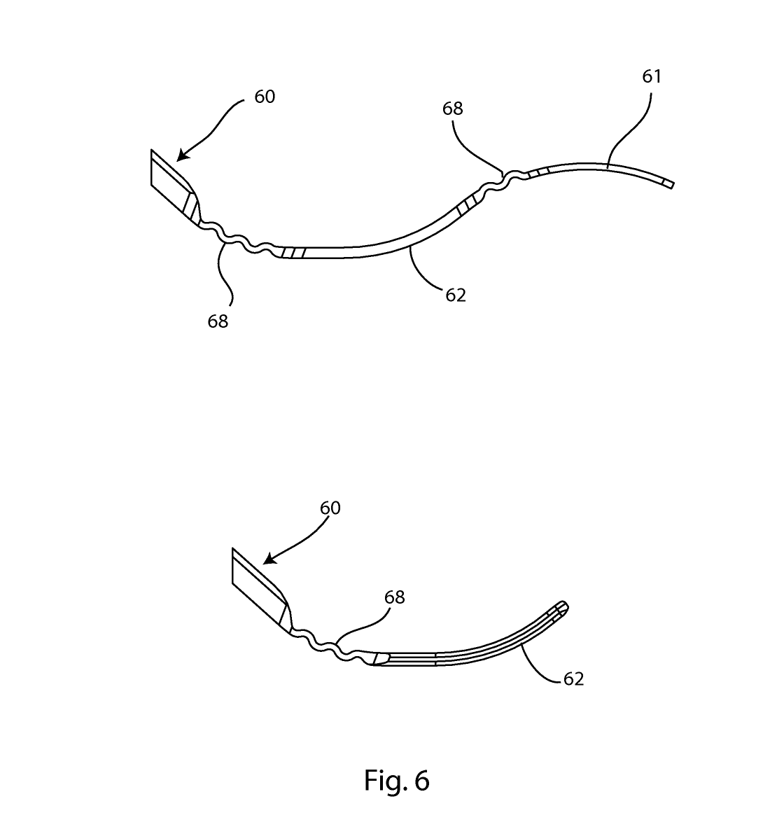

Referring to FIG. 5, a measurement device 60 may include a shaft 64 and a tip 66. The configuration in FIG. 6 illustrates a flexible measuring device that is made to replicate the desired anatomic curvature of the treatment location, such as a femoral head/neck junction. This anatomic curvature may be built into the measurement device 60 to provide a template for the user to allow the user to visually determine if the proper amount of tissue has been resected. The instrument may have an additional feature 61 to conform to a region adjacent to the treatment location, in this example the femoral head, but is not required, as shown in the right hand image of FIG. 6. The tip 66 may include one or more flexibility zones 68, here illustrated as wavy portions.

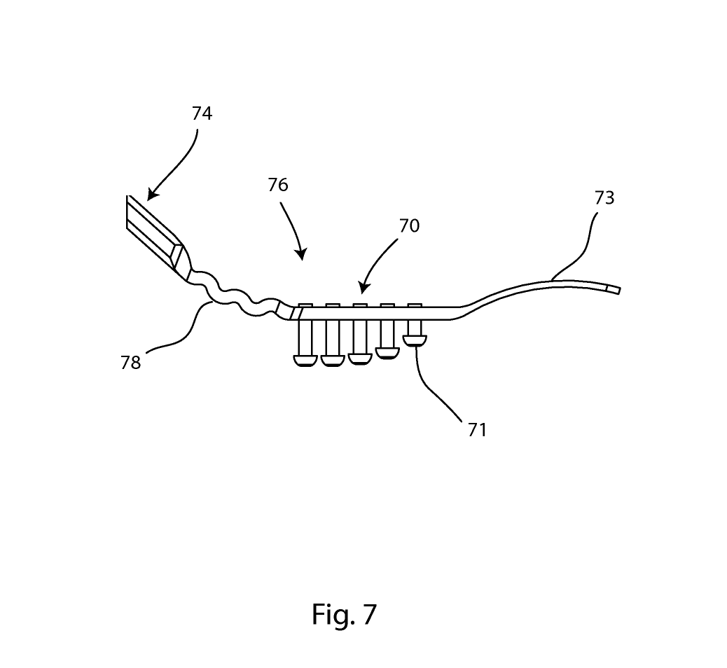

FIG. 7 depicts a tip 76 and a portion of a shaft 74 of another measurement device 70. In this example, a plurality of spring-biased indicators 71 are used to detect irregularities in the desired anatomic curvature. The ideal geometry may be indicated by the indicators being flat along the top of the instrument when the bottoms of the indicators rest on a surface. Any deviation from this flat arrangement indicates a region of tissue that requires resection in order to produce the desired curvature to the treatment region, for example to provide impingement-free motion of a joint. This measurement device 70 may allow the user to pinpoint the exact location and size of sclerotic lesions. The tip 76 may include an optional extension 73 to conform to a region adjacent to the treatment location, in this example a femoral head. The tip 76 may include one or more flexibility zones 78.

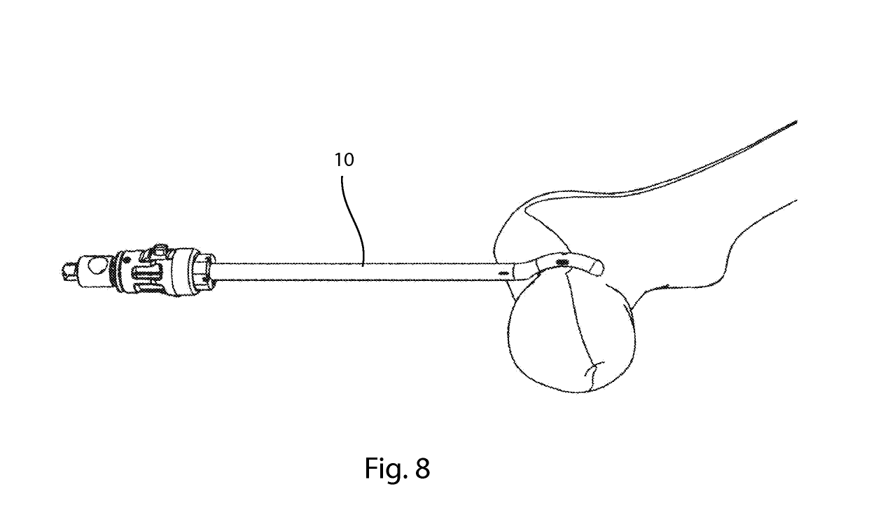

In one example, the tissue resection device 10 depicted in FIGS. 1 and 8 may have geometry specific to femoroacetabular impingement surgery to alleviate a cam impingement from an approach perpendicular to the femoral neck. Cam impingement is a type of femoroacetabular impingement in which protruding tissue on the femoral neck and/or head impinges the acetabular rim. A perpendicular approach may allow for access to a larger section of the femoral neck, which may improve the ability to sculpt the anatomy. This curved tissue removal technology may be applied to a multitude of tissue removal applications and is not limited to arthroscopic hip surgery. The tissue resection device 10 may be introduced through an access portal and directed generally perpendicular to the femoral neck, until the cutting inserts 40 engage the femoral neck, head/neck junction, or head as shown in FIG. 8. The device 10 may be swept along the femoral neck to remove asperities and restore the neck to a smooth, anatomically correct curve.

In other examples, the tissue resection devices disclosed herein may be configured to enter through different portals that are not perpendicular to the femoral neck, such as the mid-anterior portal. In some examples, the tissue resection devices may include a pivoting section (not shown) between the arcuate portion at the distal end of the tissue resection device and the hub at the proximal end of the tissue resection device. The pivoting section may be positioned at any point along the outer sheath 30 or it may be part of the arcuate portion or the hub. In other examples, the outer sheath may be permanently angled at any point and at any angle along its length. In still further examples, the outer sheath 30 may have multiple angles along its length, any of which may be permanently formed in the outer sheath 30, or achieved by including one or more pivoting sections along the length of the outer sheath. In yet further examples, the outer sheath may be permanently curved at any point and at any radius of curvature along its length. In still other examples, the outer sheath 30 may be flexible along its entire length, or at one or more points or portions along its length to allow the surgeon to bend the outer sheath 30 to any desired shape or angle during surgery.

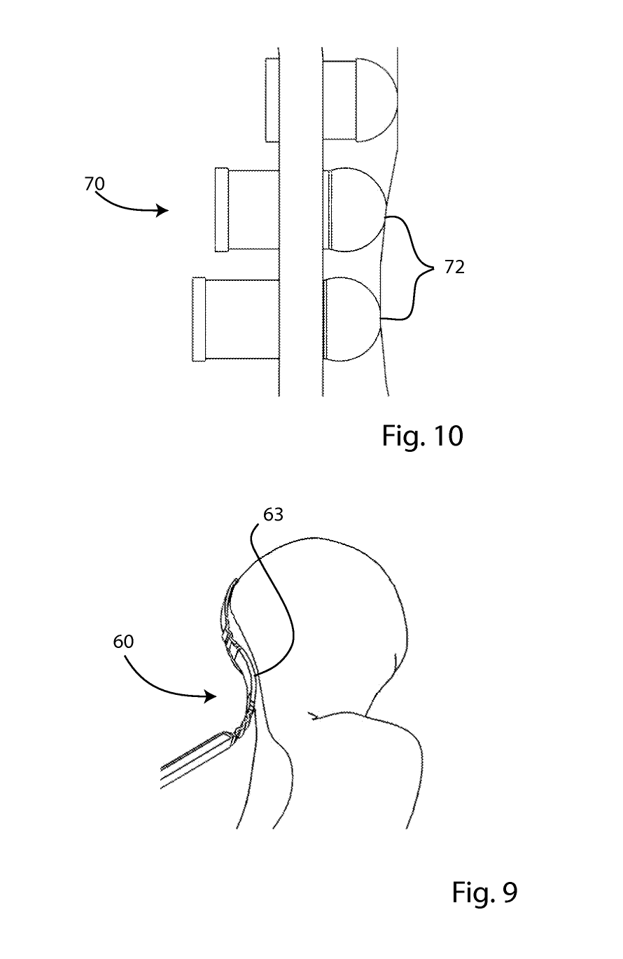

FIG. 9 shows the flexible measurement device 60 of FIG. 6 interacting with a femoral head/neck junction 63 with the rigid section 62 not aligning flat to the anatomy. This mal-alignment may indicate that tissue should be resected in this region for impingement-free motion of the hip joint. FIG. 10 depicts a similar arrangement for the device 70 of FIG. 7, and illustrates the movement of the indicators 71 where they interact with the tissue to be resected 72. The indicators have moved out of line to indicate the zone of tissue resection.

Concerning materials, in the tissue resection device 10, the cutting surfaces and housing may both be made of a biocompatible metal, such as stainless steel. The measurement instruments 60 and 70 may be constructed of a biocompatible polymeric material, such as polypropylene. Other medical grade metals or plastics may also be used, either alone or in combination. In addition, surface treatments or additives may be included to provide beneficial effects such as anti-wear or other improved mechanical properties.

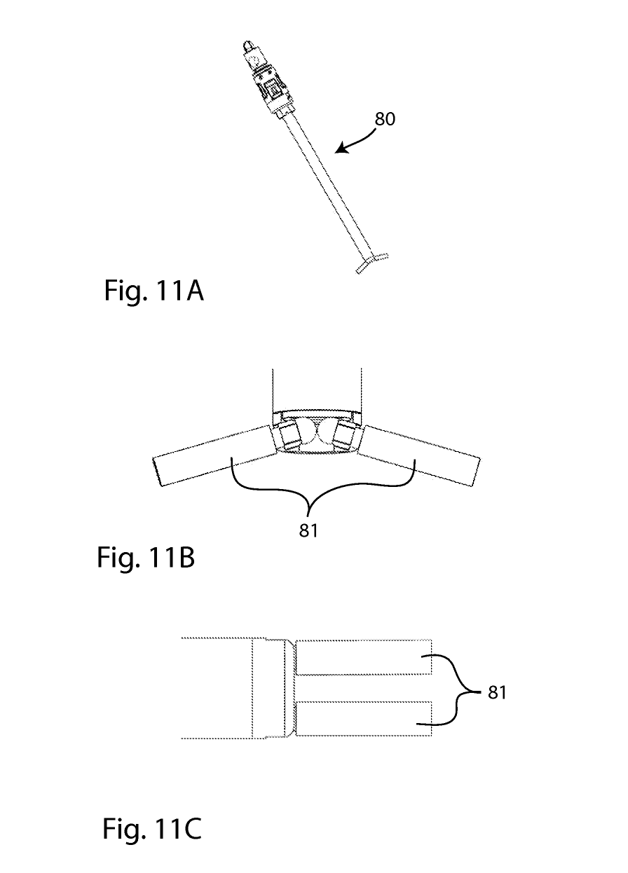

FIGS. 11A, 11B, and 11C show an expandable burr 80 for resecting tissue across a broad area. The open position (FIGS. 11A-B) allows for the cutting surfaces 81 to cover more area than the instrument initially requires in the retracted position (FIG. 11C) for delivery to the surgical site. The cutting surfaces 81 are driven to rotate by a bevel gear arrangement in a distal end of a shaft of the expandable burr 80. The cutting surfaces 81 may be roughened, textured, or fluted with straight or helical cutting flutes.

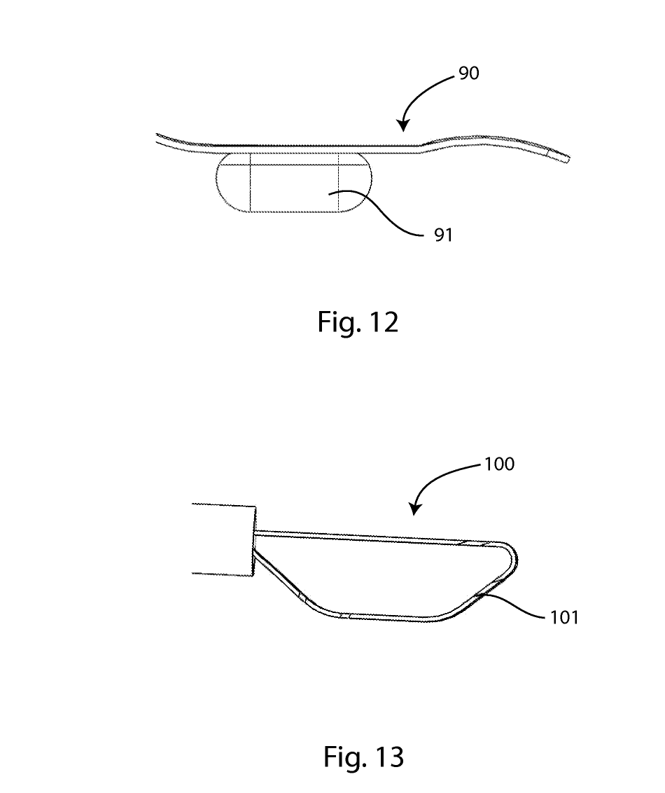

FIG. 12 shows yet another measurement device 90, which includes a bladder 91 to indicate a volumetric abnormality from the desired geometry. Volumetric changes in the bladder 91 may be read by the user by either a fluid or gas triggering a movement along a calibrated scale. The scale may be located along a shaft of the instrument. Another example of this measurement method would be a closed system with a syringe attached. When actuated, the plunger of the syringe would move per the change in volume of the bladder.

FIG. 13 illustrates yet another measurement device 100. In this example, spring deflection may be used to establish deviation from a desired geometry 101. The flexing of the instrument to accommodate abnormal anatomy may be read on a calibrated scale. The scale may be located along a shaft of the instrument.

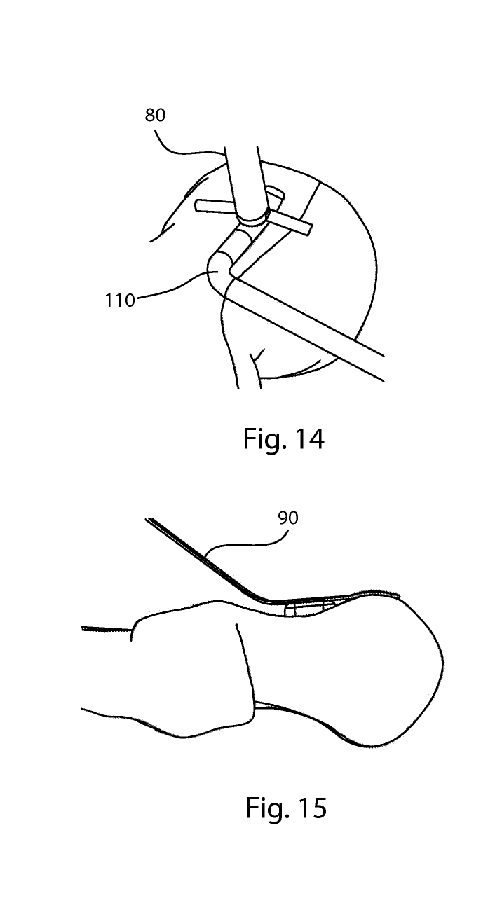

FIG. 14 shows the expandable burr 80 of FIGS. 11A-11C in use on a femoral neck with a fixed guide 110 acting as a cutting template for the burr 80. The use of a fixed template may also keep the user from resecting more tissue than desired. In this example, the expandable burr 80 slides along the fixed guide 110 with the cutting surfaces 81 extending bilaterally from the guide 110 to contact and cut tissue.

FIG. 15 shows the measurement device 90 of FIG. 12 in use against a femoral neck and head. One may appreciate that fluid or gas will move out of the bladder in response to pressure urging the bladder against a protruding tissue surface, thus producing an indication on a scale.

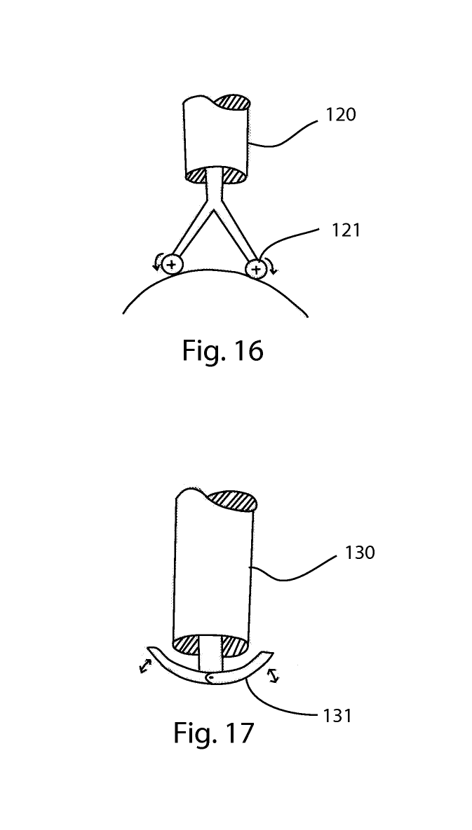

FIG. 16 shows a deployable grinder 120 which may be used to sculpt the tissue, such as bony tissue of the femoral head and neck region. This may be achieved with a plurality of rotating cutters 121. The cutters 121 may rotate in opposite directions. While two cutters are shown, more may be provided. The opposing rotations help keep the instrument easily focused on the desired region. Opposing rotation toward a midline or central axis of the grinder 120 urges debris centrally for easy removal. A depth stop (not shown) may also be included with the grinder 120 to prevent unintended tissue removal. For example, an adjustable central plunger may be provided between the cutters 121.

FIG. 17 shows a deployable grater 130 which may be inserted into the surgical space in a collapsed position. When spun, the grater may deploy cutting surfaces 131 outward to contact the tissue to be resected. A deployable cutting surface has the advantage of a minimally invasive insertion while covering a broad area once actuated.

FIGS. 20A-21 show one example of a resection device 301 having a central flexible member 308 on which cutting inserts 304 may be arranged in a stack. The central flexible member 308 in this example is hollow and has lateral holes 310 along its length. This cannulated flexible member 308 permits fluid injection or fluid suction during surgery to facilitate removal of tissue debris and keep the cutting inserts 304 from clogging up with tissue debris.

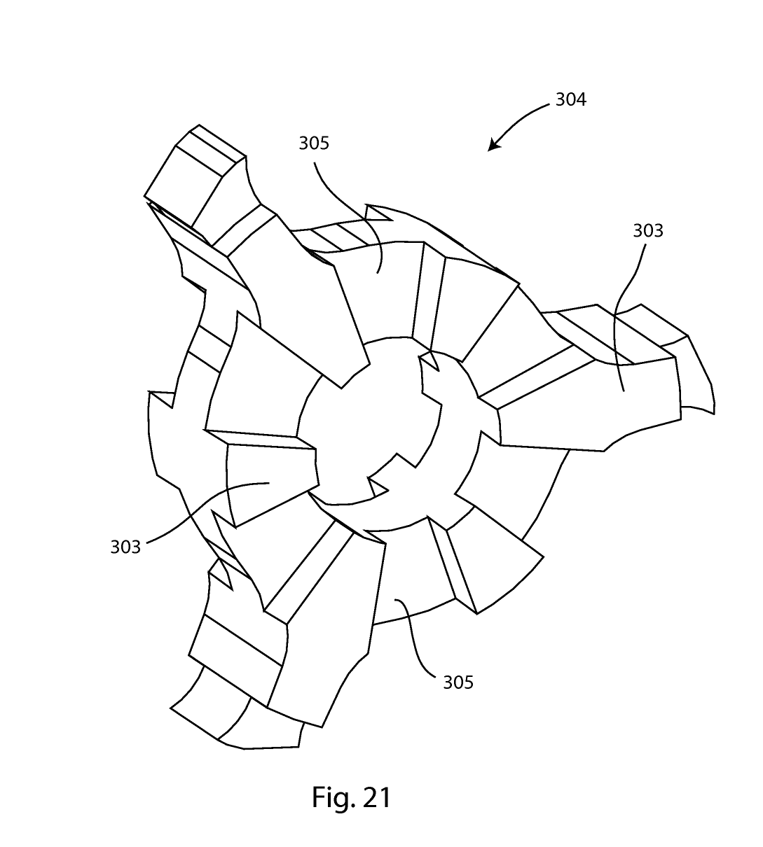

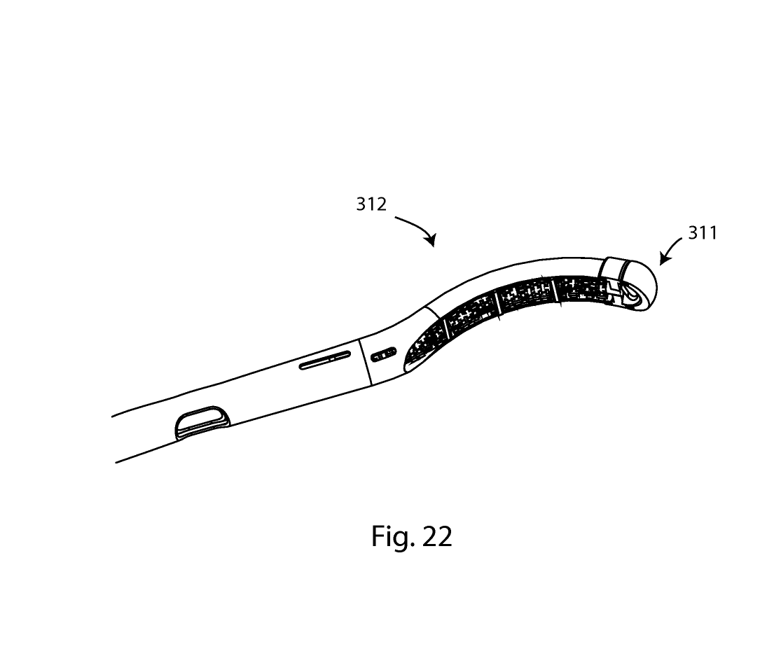

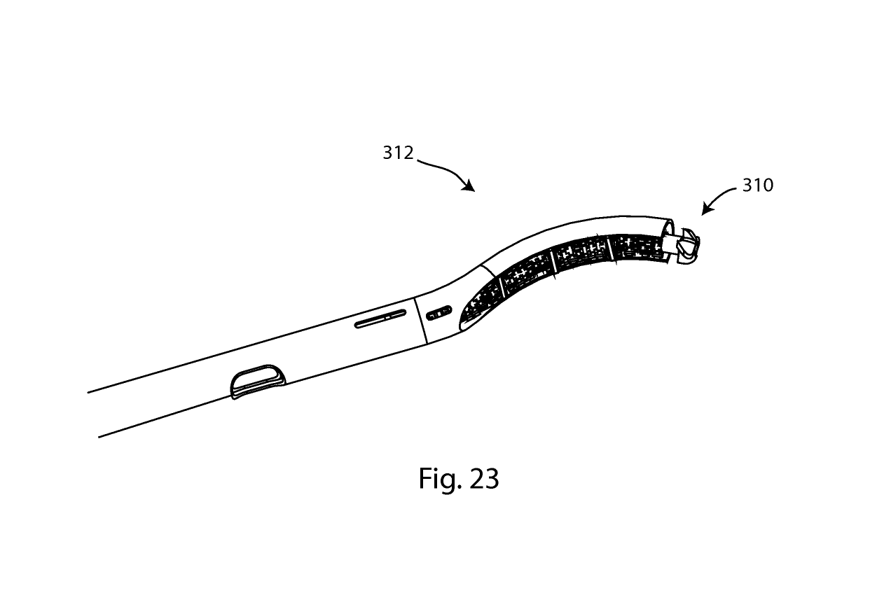

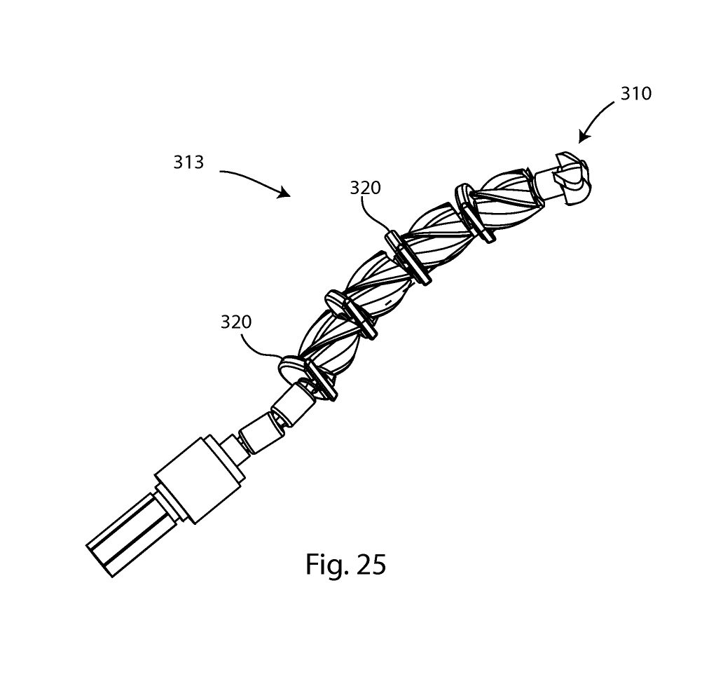

FIGS. 22-25 show examples of resection devices 312, 313 that include end-cutting elements 310 on the distal tip of the instrument to perform end cuts or resections in addition to the side cutting provided by previous examples. For example, end-cutting element 310 may allow the surgeon to resect portions of an acetabular rim to reduce “pincer-type” impingements. The curved shape of the burr may also be helpful in allowing increased visualization of a rim resection procedure. The end-cutting element 310 may be covered by a protection shield or cap 311 that may be selectively removed to perform end cutting.

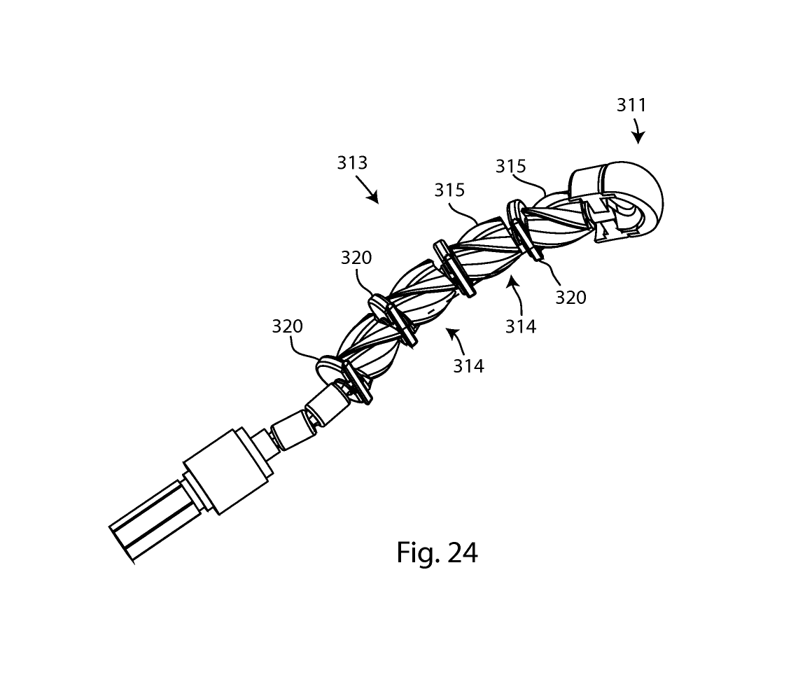

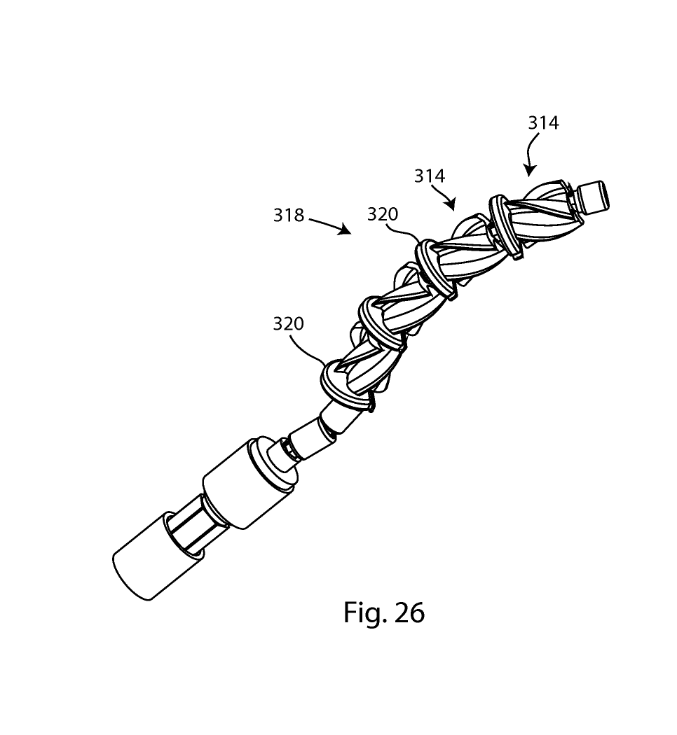

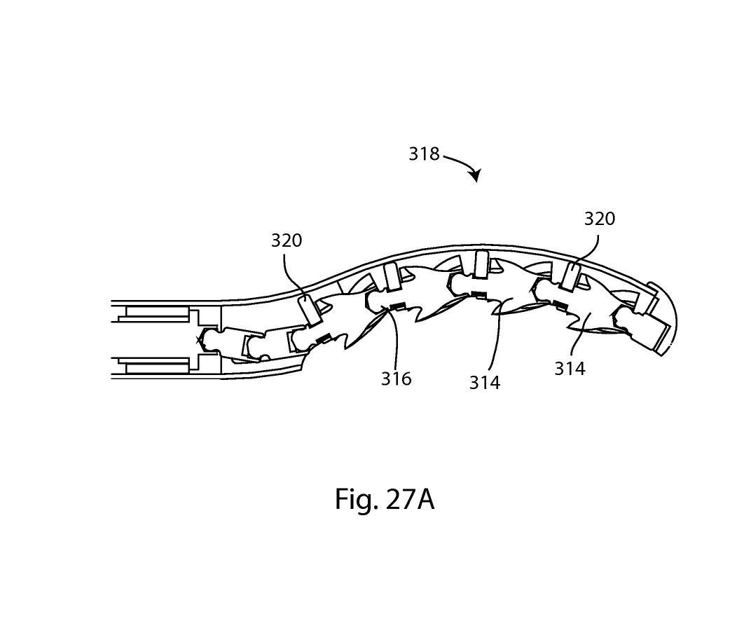

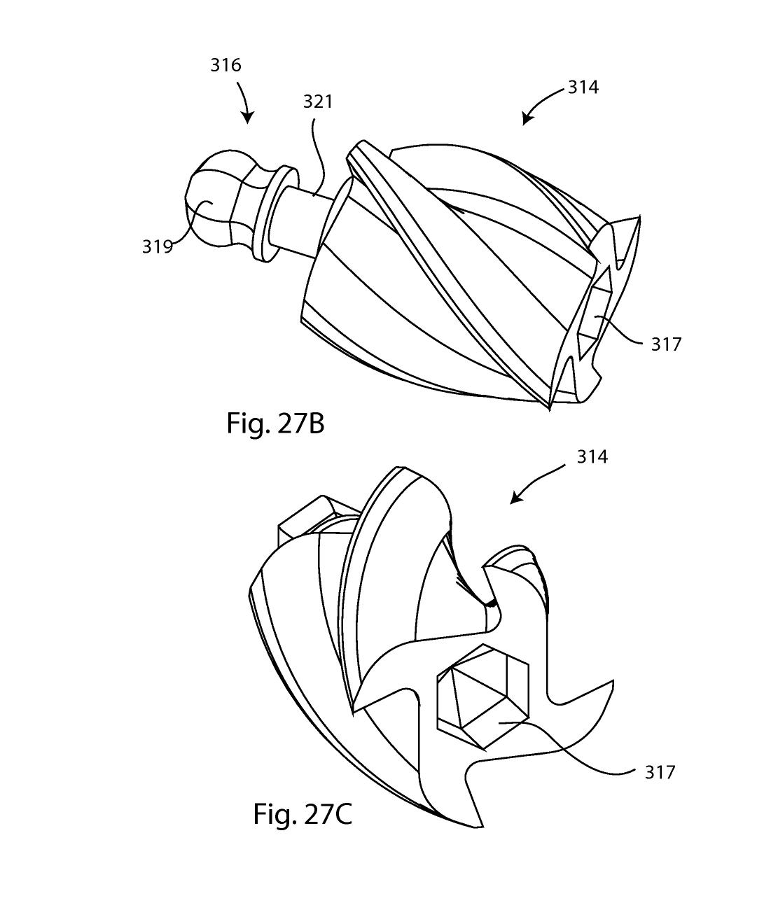

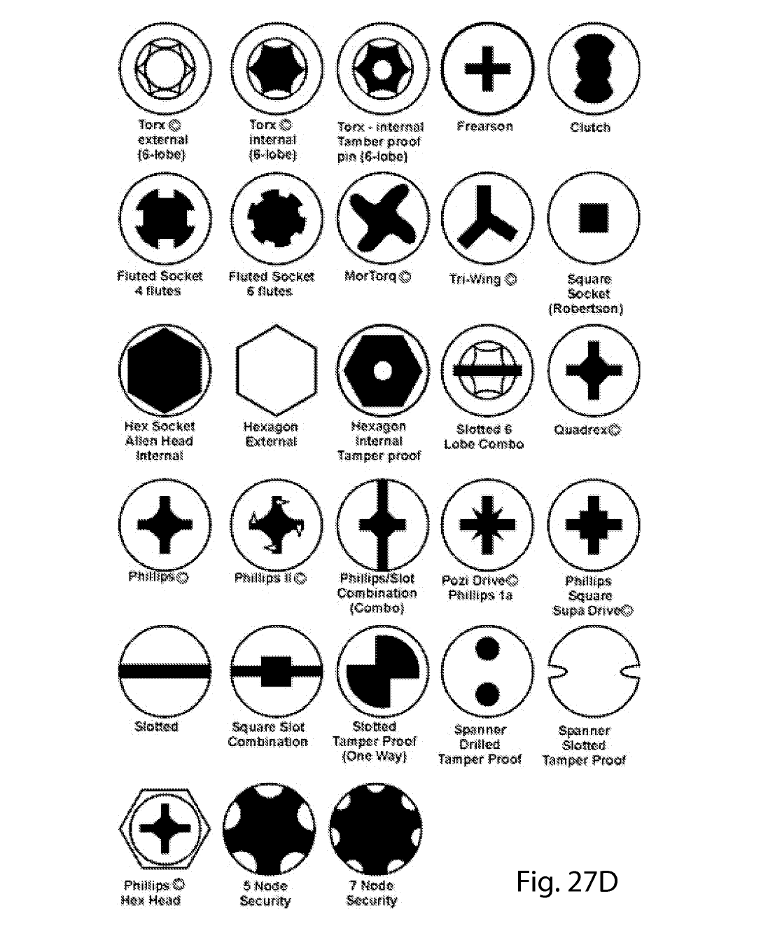

FIGS. 24-27C illustrate examples of resection devices 313, 318 that include a different style of cutting insert. In these examples, cutting inserts 314 are arranged along a curved line by connecting a series of cutting inserts 314 together. The cutting inserts 314 may have one or more propeller, helical, or fluted blades or cutting edges 315 for resecting tissue. Each cutting insert 314 may have a male 316 and female 317 mating feature at opposite ends, as may be seen in FIGS. 27B and 27C. The mating features 316, 317 are able to transmit torque between adjacent cutting inserts 314 as may be seen by the cross-section of the resection device 318 shown in FIG. 27A. Arranging the cutting inserts 314 in series creates a drivetrain capable of transmitting torque through the system. The male and female mating features may be designed to allow for misalignment in the long axis of the components, such that each cutting insert 314 may still drive adjacent cutting inserts 314 even though the rotational axes of adjacent inserts are not co-linear. This allows the burrs 314 to assume a curved path. In one example, the cutting inserts 314 are connected in series, through mating ball-hex joints 319. The ball-hex joints 319 are an example of a torque coupling which transmits torque between two adjacent cutting inserts which are set at an angle to each other. However, any suitable connection shape may be used between adjacent cutting inserts 314, including, but not limited to, the shapes illustrated in FIG. 27D.

The resection devices 313, 318 may also include bearing elements 320 that hold the cutting inserts relative to the outer housing. The bearing elements 320 may engage circular features or shafts 321 on the cutting inserts to allow the cutting inserts to rotate freely relative to the housing and maintain the axial location of the cutting inserts. The bearing elements 320 may be larger in diameter than the cutting inserts to prevent the sharp cutting surfaces of the cutting inserts from contacting the outer housing.

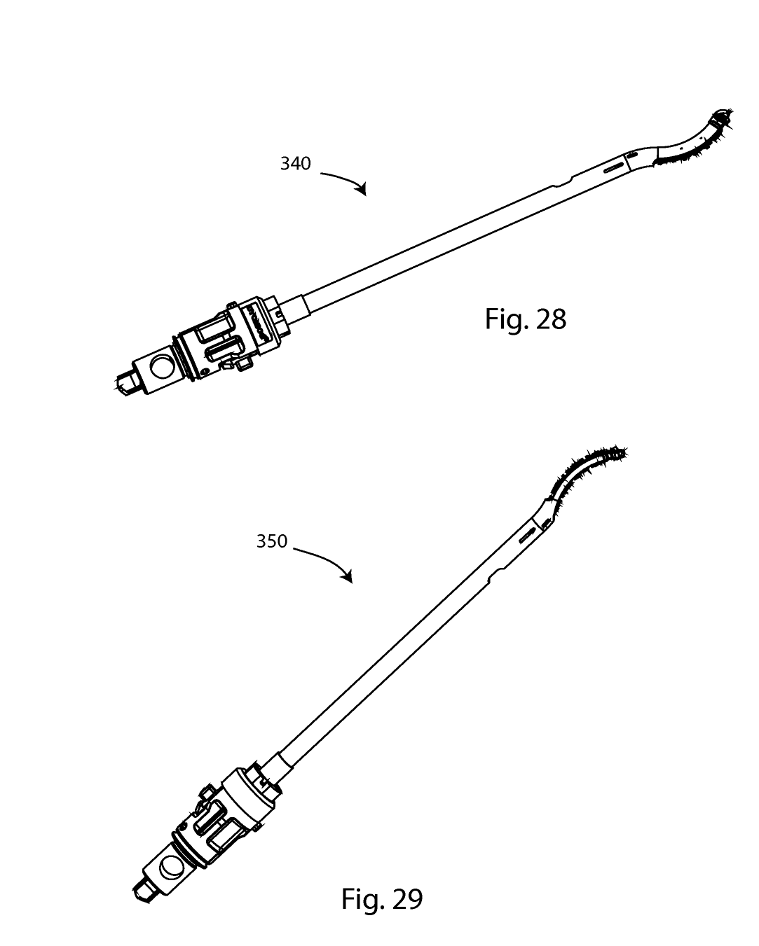

FIG. 28 illustrates a resection device 340 that has a window or cutout on the convex side of the curved portion of the resection device 340 that exposes the cutting inserts on the convex side of the outer housing. FIG. 29 illustrates a resection device 350 that has a window on the convex side of the curved portion of the resection device, as well as a window on the concave portion of the resection device. Other examples may include windows on the sides of the resection device (not shown) perpendicular to the example of FIG. 29. Still other examples may include staggered or intermittent windows along any portion of the resection device.

Any of the devices disclosed herein may include outer housings having one or more retractable and extendible portions (not shown) to selectively uncover, cover, or partially cover one or more windows to infinitely control the size and shape of the cutting surface allowed to resect tissue. These retractable and extendable portions may be remotely controlled by the surgeon during surgery by mechanical, or other means, such as a sliding tab located on the handle of the device (not shown).

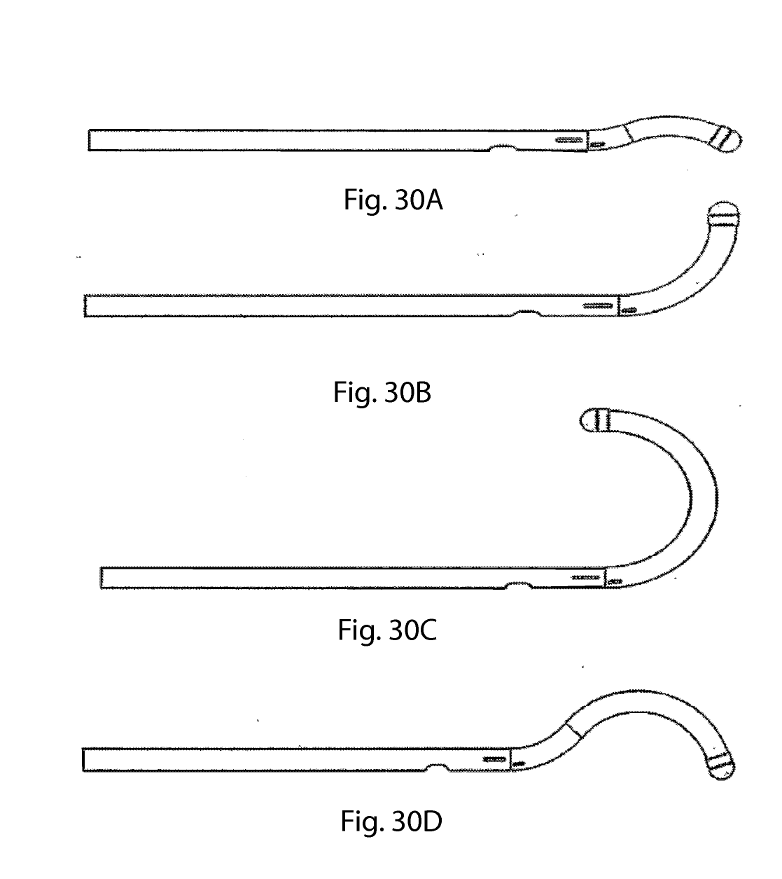

FIGS. 30A-30D show some examples of various different shapes that the resection devices of the present disclosure may assume to produce different shaped tissue resections. There are an infinite number of curves, combinations of curves, or other shapes that the resection devices of the present disclosure may assume without departing from the spirit or scope of the present disclosure.

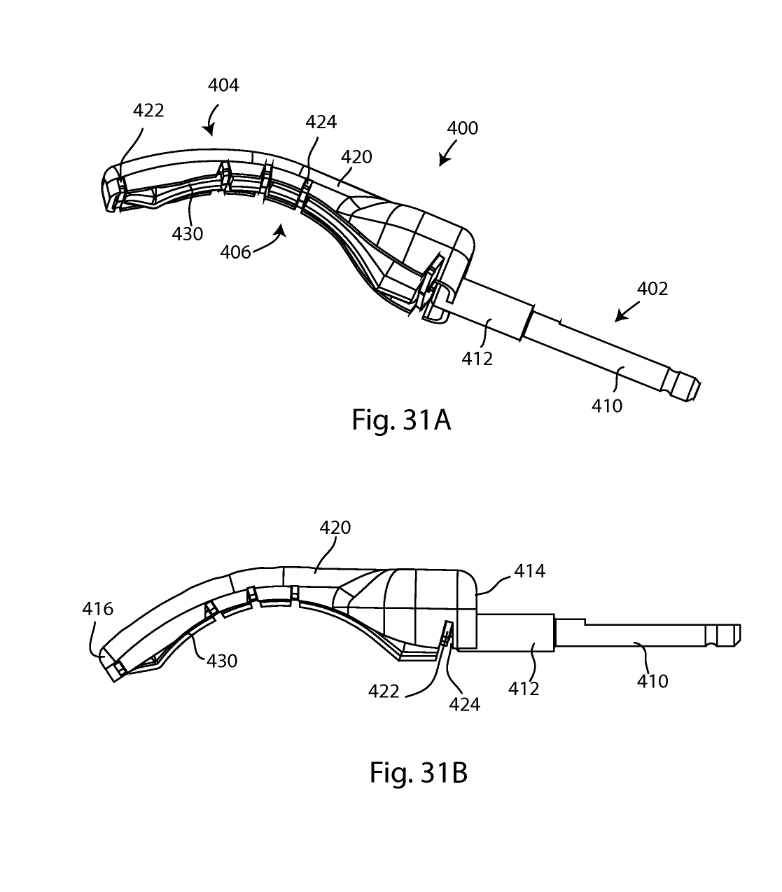

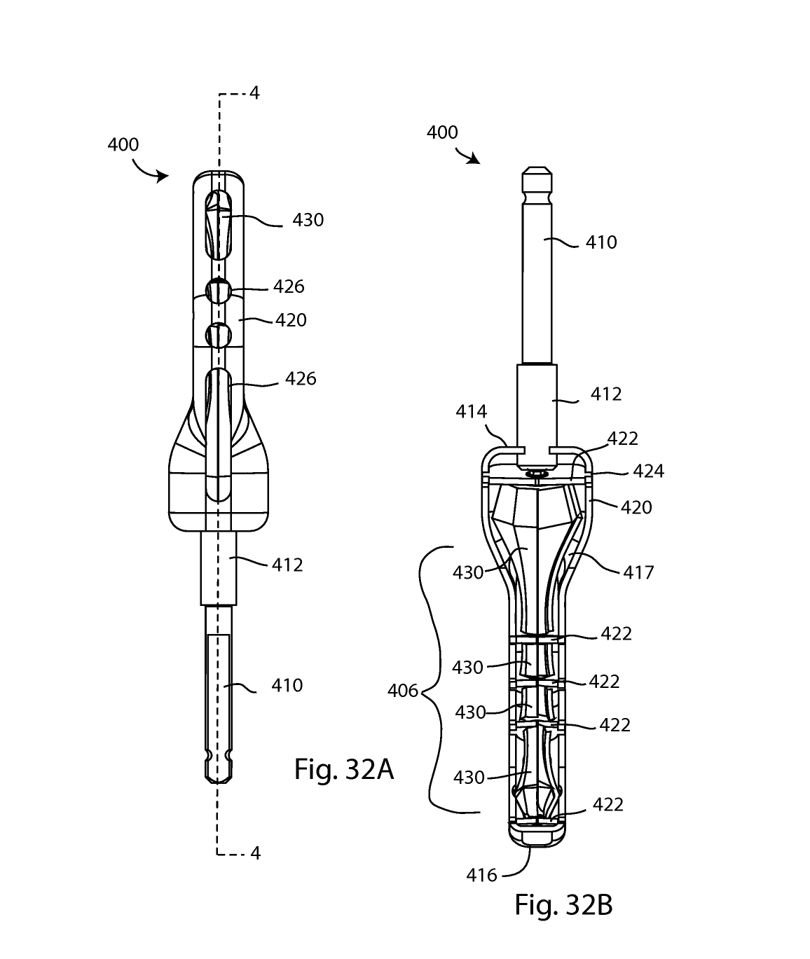

Referring to FIGS. 31A-34, a tissue resection device 400 includes a drive assembly 402, housing assembly 404, and cutting assembly 406. Tissue resection device 400 may be referred to as a burr. In some examples, the drive assembly 402 may be adapted for grasping the tissue resection device and manipulating the position of the tissue resection device in any direction or orientation. For example, the drive assembly 402 may be adapted to interact with a powered hand piece (not shown) forming a handle for grasping the tissue resection device and manipulating the position of the tissue resection device in any direction or orientation. In other examples, the drive assembly 402 may include an integral handle formed therein. In the example shown, the drive assembly includes a drive shaft 410 which has an AO connector for connection to a powered hand piece. An outer shaft 412 attaches to the housing assembly 404 to prevent rotation of the housing assembly and provide guidance. In other examples, the drive assembly may be adapted to include other functions such as suction and/or irrigation.

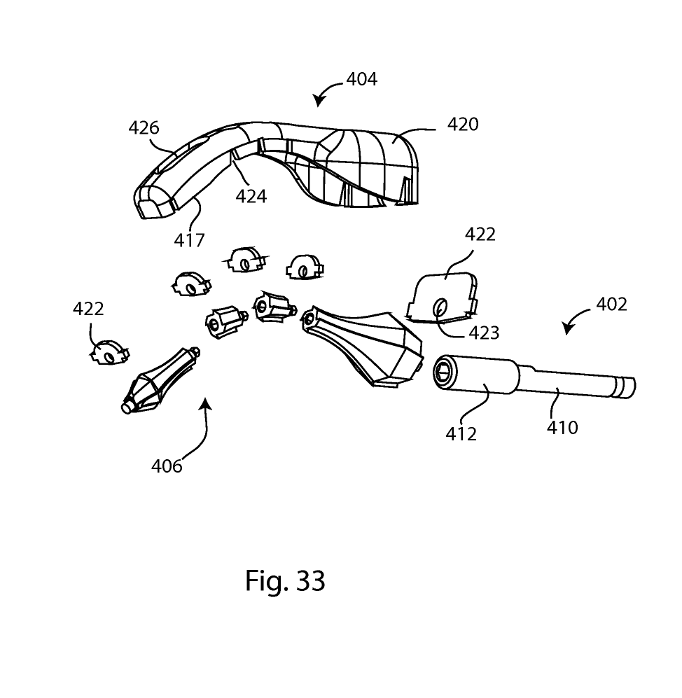

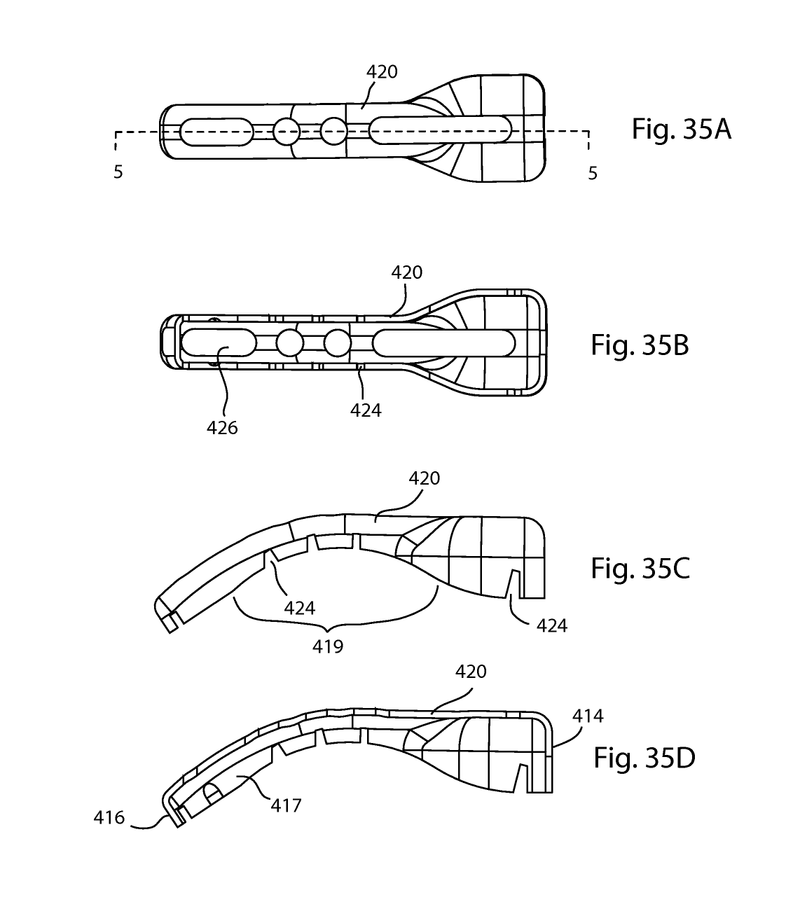

Referring to FIGS. 33 and 35A-35D, the housing assembly 404 includes an outer housing 420 and a plurality of bearings, or bushings 422. The outer housing has a proximal end 414 and a distal end 416. The housing is open on at least one side to form a cutting window 417 between the proximal and distal ends. In the example shown, the bushings are fitted into gaps 424 in the outer housing 420; they may be press-fit or snap fitted into the gaps. In other examples, the bushings may be welded to the housing, or may be formed integrally with the housing. Each bushing includes an opening 423 for receiving and guiding a cutting insert 430. The outer housing 420 may also include a plurality of fenestrations 426 through which the cutting assembly 406 may be viewed. The fenestrations 426 also decrease the weight of the device 400 and may allow for easier cleaning of the device 400. The fenestrations 426 may also serve as suction portals. The outer housing 420 has a curved shape including an arcuate portion 419 which creates a desired cutting profile; other examples may include other shapes to create other cutting profiles.

The cutting assembly 406 includes a plurality of cutting inserts 430 arranged in a sequence. Each cutting insert 430 is coupled to the next insert 430 in the sequence, to form the cutting assembly 406. The most proximal cutting insert 430 is coupled to the drive shaft 410 so that axial rotation of the drive shaft 410 transmits torque and causes the entire cutting assembly 406 to axially rotate. The cutting inserts 430 may vary in length, width and edge profile, between inserts and within any insert.

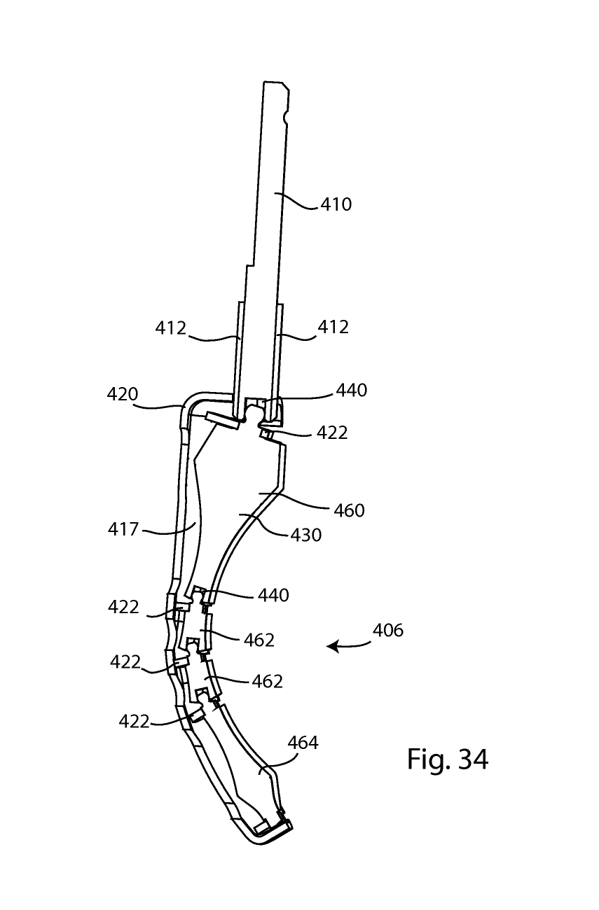

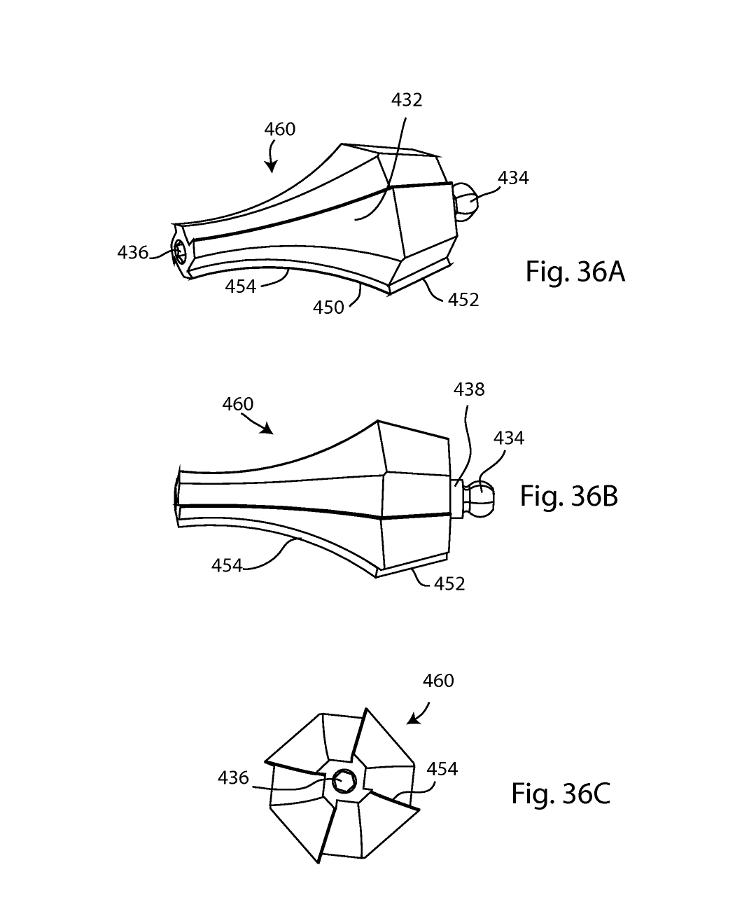

Referring to FIGS. 34 and 36A-36C, each cutting insert 430 includes a cutting body 432, a hex ball 434 and a hex ball socket 436, similar to the description above for cutting insert 314. This enables each cutting insert 430 to be operably coupled to the next cutting insert in a sequence. The hex ball 434 of one insert 430 is received in the hex ball socket 436 of the adjacent insert 430 to form a ball-hex joint 440, as seen in FIG. 34. A ball-hex joint is also formed between the drive shaft 410 and the proximal most cutting insert 430. In the examples shown, each of the ball hex joints provides angulation between adjacent inserts of up to +/−18°, although in other examples the angulation may be greater. A collar 438 separates the hex ball 434 and the cutting body 432. In other examples, each cutting insert 430 may include an axial bore extending the length of the insert to allow stringing of the inserts onto a rod or flexible member as described above. In other examples, another complementary joint such as a torx or lobed joint may be used instead of a ball-hex joint, or the shapes illustrated in FIG. 27D.

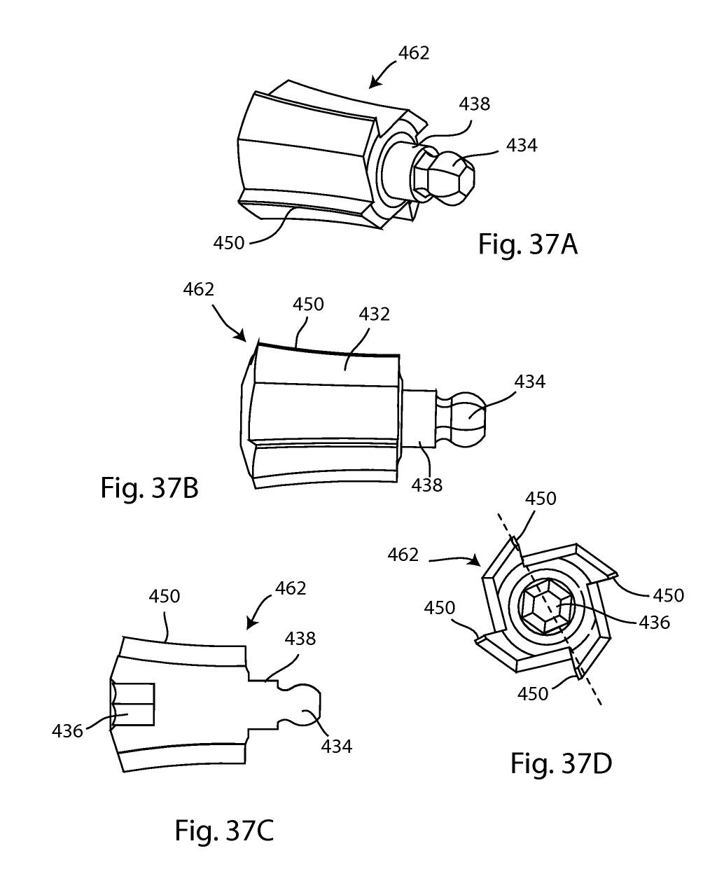

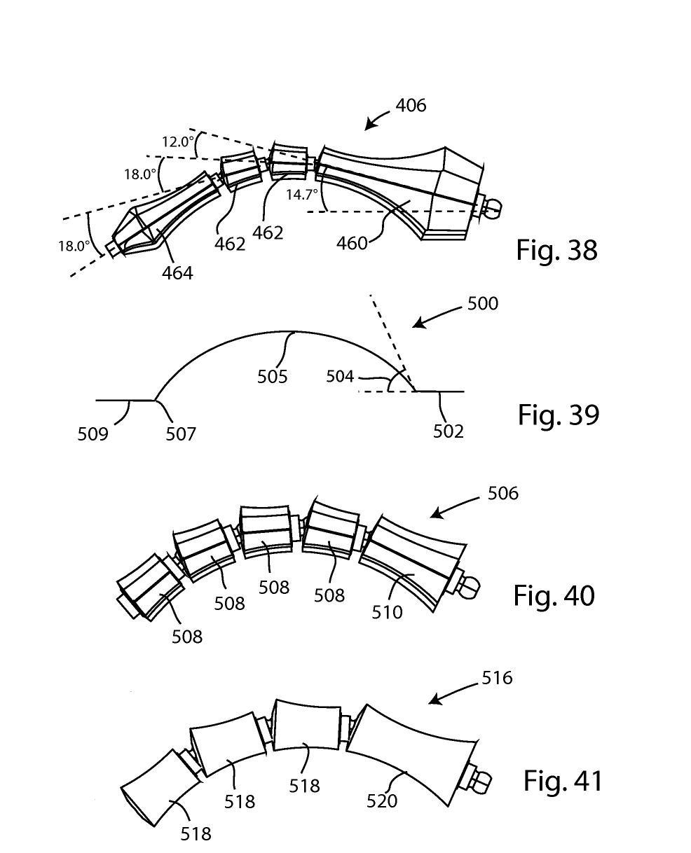

Each cutting insert 430 has at least one cutting edge 450. The cutting edges may be uniquely shaped to provide a desired cutting profile; each cutting edge 450 on an individual insert 430 may be straight, curved, spiral, or include a combination of straight, curved or spiral sections to provide the desired cutting profile. Curved sections may be convexly or concavely curved, or include a complex curve. The cutting edges 450 may be continuous, discontinuous, intersecting, or serrated. For example referring to FIGS. 36A-36C, a first cutting insert 460 includes cutting edges 450 with a straight section 452 and a curved section 454 separated by an angle; the resulting cutting profile will include a straight portion and a portion of an arc (as seen in FIG. 39). For another example, referring to FIGS. 37A-37D, a second cutting insert 462 includes cutting edges 450, each of which is slightly curved. Referring to FIGS. 34 and 38, it may be seen that cutting insert 460, two cutting inserts 462, and another cutting insert 464 are coupled in sequence to form cutting assembly 406. The diameter of each cutting insert may vary within or between each cutting section. Each cutting insert 460, 462, 464 in this example is non-cylindrical; the diameter of the cutting insert is not constant along the length of the insert. These non-cylindrical shapes contribute to the ability of the cutting assembly 406 to more closely approximate a curved profile than would cylindrical insert shapes. However, it is appreciated that any combination of variously sized and shaped cutting inserts may be coupled in sequence to form a cutting assembly.

When operatively assembled in the outer housing 420 as in FIG. 34, each cutting insert 430 is separated from the next insert by a bushing 422. The collar 438 of each insert 430 is received in the opening 423 of a bushing 422. The curvature of the outer housing 420 and the attached bushings 422 determine the final shape of the cutting assembly 406. Portions of the inserts 430 project out of the cutting window 417 of the outer housing 420. The distal end 416 of the outer housing 420 projects distally past insert 464. Referring to FIG. 39, a cutting profile 500 which may be produced by a device 400 with cutting assembly 406 is shown. The cutting profile 500 is a two-dimensional representation of the shape cut by device 400 with cutting assembly 406. Cutting profile 500 includes a first straight portion 502, a first angle 504, a first curved portion 505, a second angle 507 and a second straight portion 509. The cutting profile 500 may be suitable for preparing a talus to receive a talar component of an ankle joint prosthesis. A three-dimensional shape having an anterior-posterior profile 500 may be prepared by moving the cutting assembly 406 of device 400 medial-laterally back and forth over the bone surface as the device 400 is powered. It is appreciated that cutting inserts 460, 462 and 464 are operatively coupled together to form cutting assembly 406 which conforms to curved portion 505 without exceeding 18° between any two of the cutting inserts. It is appreciated that in other examples the degrees of angles 504, 507 may vary, as may the degree of curvature of curved portion 505. Curved portion 505 may be an arc of a circle.

In a method of use, device 400 may be used to shape a tissue surface to receive a prosthesis or other implantable member. Device 400 is operatively assembled by connecting handle assembly 402 to a powered handpiece. The distal end of device 400 is inserted into the targeted tissue area, with cutting assembly 406 directly adjacent the tissue surface to be shaped. The handpiece is powered to rotate drive shaft 410 and consequently cutting assembly 406. The device 400 is moved across the tissue surface, substantially normal to the longitudinal axis of the drive shaft 410. The device may be moved across the surface once, or back and forth, covering the same area repeatedly. When a desired amount of tissue removal is completed, the handpiece may be powered down and the device removed from the targeted tissue area. Suction may be used during or after resection to remove tissue particles. Optionally, a rongeur may be inserted into the targeted tissue area and used to remove any excess tissue and/or smooth the tissue surface.

In a method of use, device 400 may be used to prepare a talus for a talar implant. The talar implant (not shown) may have a curved attachment surface to conform closely to the anatomic geometry of the talus. By conforming to the anatomic geometry, minimal bone removal is required. Subsidence of the implant into the talus may be minimized or prevented by minimizing bone removal. However, by conforming to the anatomic geometry, this goal of minimal bone removal requires curved geometry bone preparation, which may be difficult to do. It may be difficult to get the correct curvature without leaving high or low spots on the bone. Also due to limited access between the talus and adjacent bones and tissues, it may be difficult to reach some portions of the bone to be prepared. The cutting inserts 430 on device 400 may conform to the implant profile. Thus, as the user moves the cutter two-dimensionally, medial-laterally in this example, the bone is cut to the three-dimensional implant profile. As device 400 may be powered from a single position on the anterior side, the user is able to prepare the full profile without having access issues related to reaching the inferior-posterior regions. The straight portions of cutting inserts 460, 464 allow the cutting edges to sink into the bone without the outer housing 420 making contact with the bone and preventing further depth of cut.

Referring to FIGS. 40-45, alternate examples of cutting assemblies are shown. Each cutting assembly includes a sequence of cutting inserts 430; the number of inserts may vary as may the shapes of the cutting edges of each insert.

FIG. 40 depicts a cutting assembly 506 having five cutting inserts; four are identical cutting inserts 508 and one is a relatively larger insert 510. It is appreciated that cutting assembly 506 may be assembled with housing assembly 404 with appropriate modifications to the distribution of bushings 422, and drive assembly 402, to create a tissue cutting device which may cut the same profile as device 400.

FIG. 41 depicts a cutting assembly 516 having five cutting inserts; three are identical cutting inserts 518 and one is a larger insert 520. It is appreciated that cutting assembly 516 may be assembled with housing assembly 404 with appropriate modifications to the distribution of bushings 422, and drive assembly 402, to create a tissue cutting device which may cut the same profile as device 400.

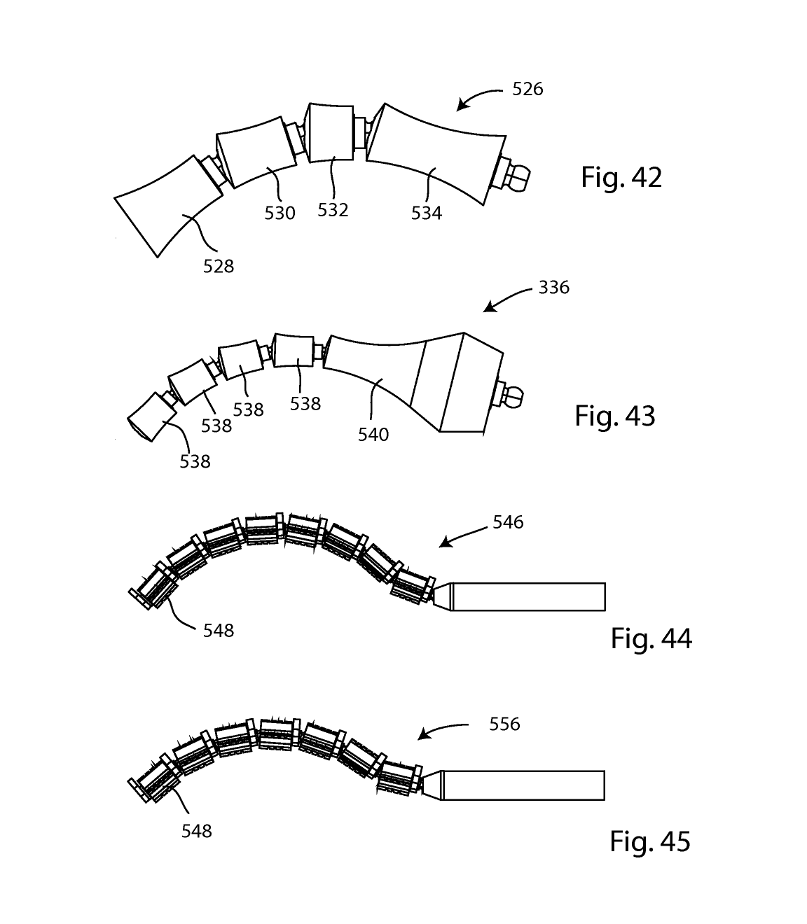

FIG. 42 depicts a cutting assembly 526 having four cutting inserts; each cutting insert 528, 530, 532, 534 differs from each other in length, width and cutting edge shape. It is appreciated that cutting assembly 526 may be assembled with housing assembly 404 with appropriate modifications to the distribution of bushings 422, and drive assembly 402, to create a tissue cutting device which may cut the same profile as device 400.

FIG. 43 depicts a cutting assembly 536 having five cutting inserts; four are identical insert 538, while cutting insert 540, is relatively longer and wider, and includes cutting edges with one curved section and two straight sections. It is appreciated that cutting assembly 536 may be assembled with housing assembly 404 with appropriate modifications to the distribution of bushings 422, and drive assembly 402, to create a tissue cutting device which may cut the same profile as device 400.

FIG. 44 depicts a cutting assembly 546 having eight identical cutting inserts 548. It is appreciated that cutting assembly 546 may be assembled with housing assembly 404 with appropriate modifications to the distribution of bushings 422, and drive assembly 402, to create a tissue cutting device which may cut the same profile as device 400. Cutting inserts 548 may be cylindrical and include serrated or scalloped cutting edges and may produce a roughened or ridged tissue surface. In some examples, serrations may be aligned or misaligned in order to leave a smooth or roughened surface as desired.

FIG. 45 depicts a cutting assembly 556 having seven identical cutting inserts 558. It is appreciated that cutting assembly 556 may be assembled with housing assembly 404 with appropriate modifications to the distribution of bushings 422, and drive assembly 402, to create a tissue cutting device which may cut the same profile as device 400. Cutting inserts 558 may be cylindrical and include serrated or scalloped cutting edges and may produce a roughened or ridged tissue surface.

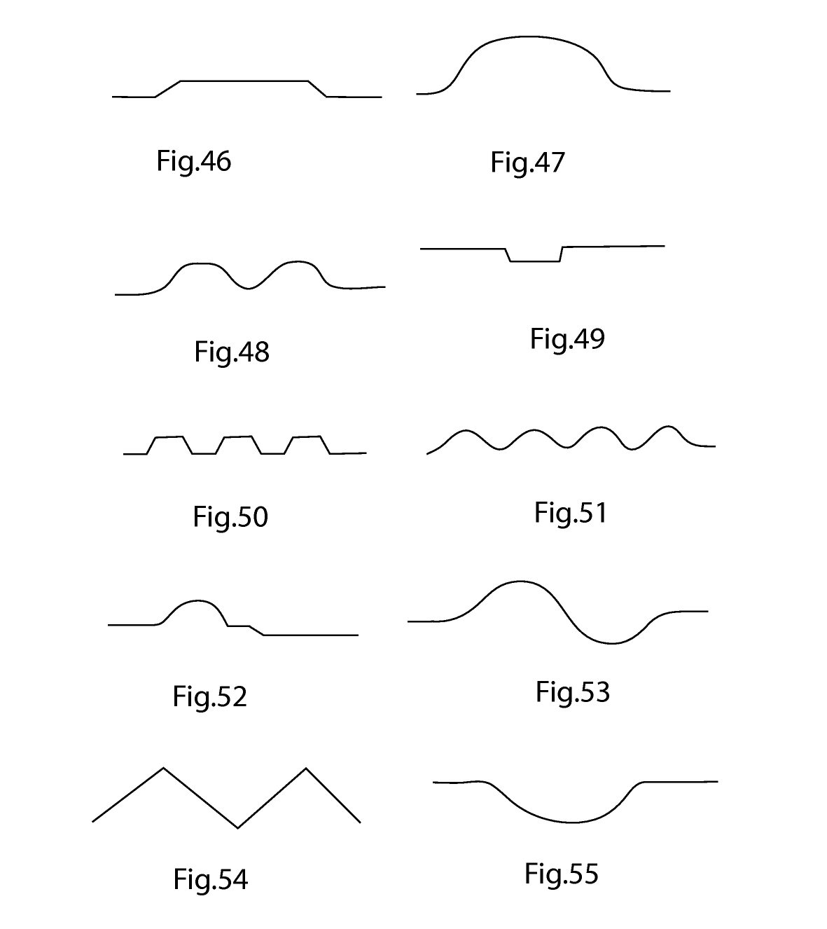

FIGS. 46-55 depict other cutting profiles which may be created by the instruments and methods disclosed herein. For each profile shown, a cutting member may be assembled comprising one or more cutting inserts as disclosed herein. The cutting inserts have straight cutting edges, curved cutting edges, or a combination thereof to create each profile. It is also appreciated that other profiles not specifically shown may be created using the instruments and methods disclosed herein. For example, the shapes shown in the various profiles of FIGS. 39 and 46-55 may be mixed and matched to create other profiles attainable by the instruments and methods disclosed herein.

The components disclosed herein may be fabricated from metals, alloys, polymers, plastics, ceramics, glasses, composite materials, or combinations thereof, including but not limited to: PEEK, titanium, titanium alloys, commercially pure titanium grade 2, ASTM F67, Nitinol, cobalt chrome, stainless steel, ultra high molecular weight polyethylene (UHMWPE), biocompatible materials, and biodegradable materials, among others. Different materials may be used for different parts. Different materials may be used within a single part. Any component disclosed herein may be colored, coded or otherwise marked to make it easier for a user to identify the type and size of the component, the setting, the function(s) of the component, and the like.

It should be understood that the present systems, kits, apparatuses, and methods are not intended to be limited to the particular forms disclosed. Rather, they are to cover all combinations, modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), '7include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

In the foregoing Detailed Description, various features are grouped together in several examples for the purpose of streamlining the disclosure. This method of disclosure is not to be interpreted as reflecting an intention that the examples of the invention require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example.

1. A talar resection device, the device comprising:
a drive shaft;
a housing extending between proximal and distal ends, a portion of the housing extending along an arcuate path forming an arcuate housing portion, the housing comprising a housing window; and
a plurality of cutting inserts coupled together in a sequence extending along the arcuate path and partially received in the housing window, wherein the plurality of cutting inserts comprises a cutting profile, wherein the cutting profile comprises a first straight portion and a curved portion.

2. The device of claim 1, where in the cutting profile further comprises a second straight portion.

3. The device of claim 1, wherein a proximal-most cutting insert is coupled to the drive shaft.

4. The device of claim 1, wherein each cutting insert comprises at least one cutting edge and wherein each cutting insert is non-cylindrical.

5. The device of claim 3, wherein the plurality of cutting inserts comprise a cutting member, wherein rotation of the drive shaft rotates the cutting member.

6. The device of claim 3, wherein each cutting insert is coupled to the adjacent cutting insert in the sequence.

7. The device of claim 6, wherein each of the plurality of cutting inserts comprises engagement features comprising complementary protrusions and depressions.

8. The device of claim 3, wherein each cutting insert has a longitudinal axis, wherein a first angle is formed between the longitudinal axes of adjacent cutting inserts in the sequence when the cutting inserts are operatively assembled in the housing.

9. The device of claim 3, wherein each cutting insert has a longitudinal axis and the drive shaft has a longitudinal axis, wherein a second angle is formed between the longitudinal axis of the drive shaft and the longitudinal axis of the proximal most cutting insert when the drive shaft and proximal most cutting insert are operatively assembled in the housing.

10. The device of claim 1, wherein the cutting profile further comprises a first angle between the first straight portion and the curved portion and a second angle between the curved portion and the second straight portion.

11. The device of claim 1 further comprising an outer shaft attached to the housing to prevent rotation of the housing.

12. A talar bone burr, the burr comprising:
a drive shaft;
a housing extending between proximal and distal ends, a portion of the housing extending along a curved path forming a curved casing portion, the housing comprising a housing window;
a plurality of bushings fitted into the housing; and
a plurality of cutting inserts coupled together in a sequence extending along the curved path and partially received in the housing window, a proximal-most cutting insert coupled to the drive shaft, wherein each cutting insert comprises at least one cutting edge and wherein each cutting insert is differently shaped than each of the other cutting inserts.

13. The burr of claim 12, wherein each of the plurality of cuttings inserts is sized relative to the proximity and placement of the plurality of bushings.

14. The burr of claim 13, wherein each of the plurality of bushings comprises an opening for receiving a portion of the cutting insert.

15. The burr of claim 12, wherein the plurality of cutting inserts comprise a cutting member, wherein rotation of the drive shaft rotates the cutting member.

16. The burr of claim 12, wherein each cutting insert is coupled to the adjacent cutting insert in the sequence through engagement features comprising complementary protrusions and depressions.

17. The burr of claim 12, wherein each cutting insert has a longitudinal axis, wherein the longitudinal axes of adjacent cutting inserts in the sequence are non-parallel when the cutting inserts are operatively assembled in the housing.

18. The burr of claim 12, wherein each cutting insert has a longitudinal axis and the drive shaft has a longitudinal axis, wherein the longitudinal axis of the drive shaft and the longitudinal axis of the proximal most cutting insert are non-parallel when the drive shaft and proximal most cutting insert are operatively assembled in the housing.

19. The burr of claim 12, wherein the plurality of cutting inserts comprise a cutting profile wherein the cutting profile comprises a first straight portion, a curved portion and a second straight portion.

20. A method for resecting a talar bone to receive an implant, the method comprising:
inserting a portion of a talar bone resection device into the patient's body;
engaging the talar bone with the talar bone resection device, the talar bone resection device comprising a drive shaft, a housing extending between proximal and distal ends, and a cutting assembly, a portion of the housing extending along an arcuate path forming an arcuate housing portion, the housing comprising a housing window,
wherein the cutting assembly further comprises a plurality of cutting inserts coupled together in a sequence extending along the arcuate path and partially received in the housing window, wherein the plurality of cutting inserts comprises a cutting profile wherein the cutting profile comprises a first straight portion, a curved portion and a second straight portion;
powering the talar bone resection device; and
moving the cutting assembly of the device in a medial-lateral direction over the bone surface.
